(19) United States
(12) Patent Application Publication
(10) Pub. No.: US 2023/0046078 A1
(43) Pub. Date: Feb. 16, 2023

(54) METHODS OF USING SUBSTITUTED FLAVONES FOR TREATING BONE DISORDERS

(71) Applicant: EMORY UNIVERSITY, Atlanta, GA (US)

(72) Inventor: Keqiang Ye, Lilburn, GA (US)

(73) Assignee: EMORY UNIVERSITY, Atlanta, GA (US)

(21) Appl. No.: 17/866,008

(22) Filed: Jul. 15, 2022

Related U.S. Application Data
(60) Provisional application No. 63/222,289, filed on Jul. 15, 2021.

(51) Int. Cl. A61K 31/352; A61P 19/08

(52) U.S. Cl. CPC A61K 31/352; A61P 19/08

(57) ABSTRACT

Provided herein are methods of using 4-oxo-2-phenyl-4H-chromene-7,8-diyl bis(methylcarbamate) and derivatives thereof for treating or preventing a bone disorder. The methods include administering to the subject an effective amount of a compound of Formula I or a pharmaceutical composition as described herein. The bone disorder can include, for example, osteoporosis. Also provided herein are methods of inhibiting asparagine endopeptidase (AEP) activity in a cell and methods of promoting bone growth, increasing bone density, or increasing bone strength in a subject. Novel mechanisms of action in treating and preventing bone disorders are provided herein.

### CROSS-REFERENCE TO RELATED APPLICATION

This application claims the benefit of U.S. Provisional Application No. 63/222,289, filed Jul. 15, 2021, which is incorporated herein by reference in its entirety.

### STATEMENT REGARDING FEDERALLY FUNDED RESEARCH

This invention was made with government support under Grant No. RF1 AG051538 awarded by the National Institutes of Health (NIH). The government has certain rights in the invention.

### BACKGROUND

Brain-derived neurotrophic factor (BDNF) belongs to the family of neurotrophins that play essential roles in the central nervous system (CNS) and are mainly expressed in central and peripheral neuronal tissues. However, BNDF is also synthesized and released from non-neuronal cells such as fibroblasts, osteoblasts, endothelial cells, monocytes, and mast cells. Plasma BDNF levels are increased in individuals with osteoarthritis compared to healthy individuals. BDNF is involved in osteoblast cell differentiation and stimulates bone/cementum-related proteins including alkaline phosphatase (ALP), bone morphogenetic protein-2 (BMP-2), and osteopontin (OPN) expression in cementoblasts. Both BDNF and its TrkB receptor are present at various stages of the bone formation process, and they are upregulated in human osteoblasts and implicated in fracture healing. BDNF strongly elevates mRNA expression of the osteoblast differentiation marker, osteocalcin, in the osteoblast-lineage cell MC3T3-E1. Additionally, BDNF stimulates cell differentiation and promotes new bone formation and maturation.

AEP (asparaginyl endopeptidase, also known as legumain with gene name: LGMN) is a broadly expressed endo-lysosomal cysteine protease that is secreted as inactive pro-zymogen (56 kDa). The inactive pro-zymogen is processed into an enzymatically active 36 kDa mature form and a 17 kDa C-terminal inhibitory fragment. Strikingly, the C-terminal truncate inhibits osteoclast differentiation through binding to an uncharacterized receptor. Active AEP inhibits osteoblast differentiation and in vivo bone formation through degradation of the bone matrix protein fibronectin. During development, AEP-deficient zebrafish exhibit precocious bone formation and mineralization. Human bone marrow stromal cells (hBMSCs) are non-hematopoietic multipotent cells capable of differentiation into mesodermal cell types such as osteoblasts and adipocytes. Markedly, AEP regulates the lineage commitment of hBMSCs. AEP is abnormally expressed and displays aberrant subcellular localization in the bone of individuals with postmenopausal osteoporosis.

### SUMMARY

The present disclosure relates to flavone derivatives, such as those described by the formula provided herein, and to pharmaceutical compositions and methods related thereto. The disclosure further relates to methods of treating or preventing bone disorders, such as osteoporosis, by administering an effective amount of a pharmaceutical composition comprising compounds disclosed herein to a subject in need thereof.

Provided herein is a method of treating or preventing a bone disorder in a subject, comprising administering to the subject an effective amount of a compound of Formula I

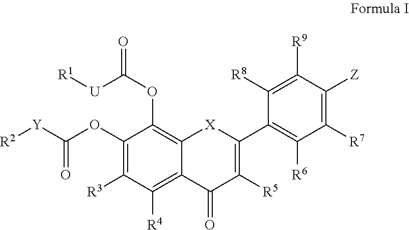

or a salt thereof, wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, U and Y are independently NH or N-alkyl. Optionally, U and Y are O. Optionally, R¹ and R² are alkyl.

Optionally, the compound is selected from the group consisting of 4-oxo-2-phenyl-4H-chromene-7,8-diyl bis(methylcarbamate), 4-oxo-2-phenyl-4H-chromene-7,8-diyl dipropionate, 4-oxo-2-phenyl-4H-chromene-7,8-diyl bis(2,2-dimethylpropanoate), diethyl (4-oxo-2-phenyl-4H-chromene-7,8-diyl) dicarbonate, 4-oxo-2-phenyl-4H-chromene-7,8-diyl bis(ethylcarbamate), 4-oxo-2-phenyl-4H-chromene-7,8-diyl bis(dimethylcarbamate), and 4-oxo-2-phenyl-4H-chromene-7,8-diyl bis(3-methylbutanoate) or salts thereof.

Optionally, the bone disorder comprises a bone disorder associated with abnormally high bone catabolism (e.g., hyperparathyroidism), a metabolic bone disease (e.g., osteoporosis, osteomalacia, rickets, renal osteodystrophy, osteopenia, Paget's disease, and osteogenesis imperfecta) or a metastatic bone disease.

The compound of Formula I is optionally administered with an additional active agent, wherein the additional active agent comprises an anti-osteoporosis agent. The anti-osteoporosis agent optionally comprises one or more of a bisphosphonate, a RANK-L inhibitor, a parathyroid hormone or parathyroid hormone analog, a therapeutic antibody (e.g., a monoclonal antibody, a chimeric antibody, a humanized antibody, or a human antibody or binding fraction thereof), estrogen or estrogen analog, and calcitonin.

Optionally, the compound of Formula I is administered orally. Optionally, the subject has or is at risk of developing a bone disorder or experiencing bone loss.

Optionally, the disclosure relates to a method of inhibiting asparagine endopeptidase (AEP) activity in a cell, comprising contacting the cell with an effective amount of a compound represented by the structure of Formula I

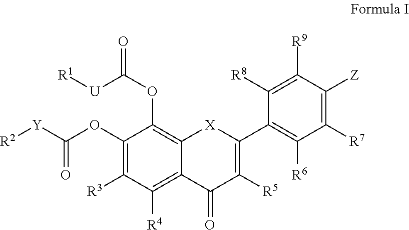

or a salt thereof, wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, the compound is selected from the group consisting of 4-oxo-2-phenyl-4H-chromene-7,8-diyl bis(methylcarbamate), 4-oxo-2-phenyl-4H-chromene-7,8-diyl dipropionate, 4-oxo-2-phenyl-4H-chromene-7,8-diyl bis(2,2-dimethylpropanoate), diethyl (4-oxo-2-phenyl-4H-chromene-7,8-diyl) dicarbonate, 4-oxo-2-phenyl-4H-chromene-7,8-diyl bis(ethylcarbamate), 4-oxo-2-phenyl-4H-chromene-7,8-diyl bis(dimethylcarbamate), and 4-oxo-2-phenyl-4H-chromene-7,8-diyl bis(3-methylbutanoate) or salts thereof.

Optionally, the subject to be treated has or is at risk of developing a bone disorder or experiencing bone loss.

The contacting step can be performed in vivo or in vitro. The contacting step can increase osteoprotegerin (OPG) levels in the cell as compared to an untreated (i.e., control) cell and/or can inhibit RANK-L-induced osteoclastogenesis in the cell.

Optionally, the contacting in vivo reduces trabecular bone loss induced by ovariectomy (OVX).

Further provided is a method of promoting bone growth, increasing bone density, or increasing bone strength in a subject, comprising administering to the subject an effective amount of a compound of Formula I

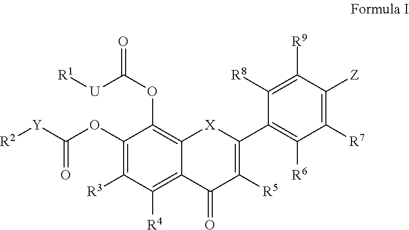

or a salt thereof, wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, the disclosure relates to a use of a compound of Formula I or a salt thereof in the preparation of a drug for treating or preventing a bone disorder,

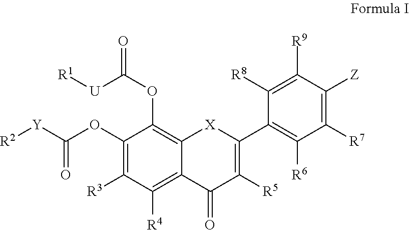

wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, the disclosure relates to a use of a compound of Formula I or a salt thereof in the preparation of a drug for inhibiting asparagine endopeptidase (AEP) activity in a cell,

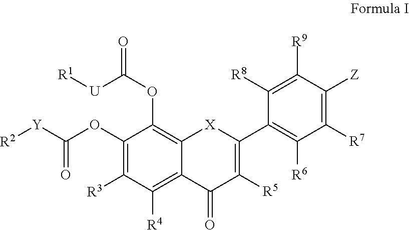

wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, the disclosure relates to a use of a compound of Formula I or a salt thereof in the preparation of a drug for promoting bone growth, increasing bone density, or increasing bone strength,

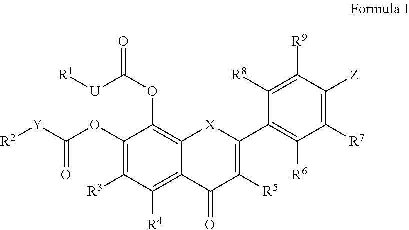

wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, the disclosure relates to a compound of Formula I or a salt thereof for use in treating or preventing a bone disorder,

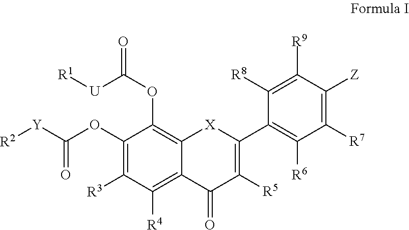

wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, the disclosure relates to a compound of Formula I or a salt thereof for use in inhibiting asparagine endopeptidase (AEP) activity in a cell,

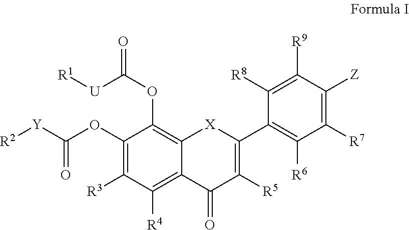

wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

Optionally, the disclosure relates to a compound of Formula I or a salt thereof for use in promoting bone growth, increasing bone density, or increasing bone strength,

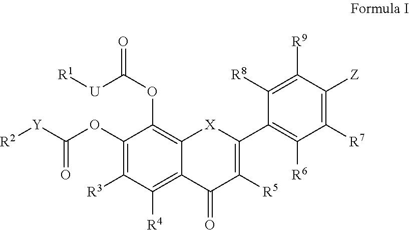

wherein X is O, S, or NH; U and Y are each independently O, S, NH, Nalkyl, or CH₂; Z is hydrogen, amino, diaminoalkyl, or heterocyclyl optionally substituted with one or more, the same or different, R¹⁵; R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵; R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵; each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

The details of one or more embodiments are set forth in the drawings and the description below. Other features, objects, and advantages will be apparent from the description and drawings, and from the claims.

### BRIEF DESCRIPTION OF THE DRAWINGS

FIG. 1, Panel A, is a photographic image showing the uterine morphology of wild-type (WT) mice with (OVX) or without (Sham) ovariectomy. FIG. 1, Panel B, is a graph depicting the weight of the uterus of the wild-type mice with (OVX) or without (Sham) ovariectomy. FIG. 1, Panel C is a graph of the body weight of WT and BDNF+/−mice after sham or ovariectomy surgery followed by treatment with Compound R13 or vehicle. Data are shown as mean±SEM, where n=8 mice per group.

FIG. 2, Panel A, is a collection of images of the femoral indices of trabecular bone structure measured by in vitro μCT scans of wild-type (AEP WT) and AEP knockout (AEP KO) mice with (OVX) and without (Sham) ovariectomy. FIG. 2, Panel B, is a set of graphs showing μCT scanning measurements of trabecular bone volume fraction (BV/TV), connectivity density (Conn.D), structure model index (SMI), trabecular number (Tb.N), trabecular spacing (Tb.Sp.), and trabecular thickness (Tb.Th.) in wild-type and knock-out animals with and without ovariectomy. Data are shown as mean±SEM, n=5 mice per group for the AEP WT sham group, n=6 mice per group for the AEP KO group, and n=7 mice per group for AEP WT and AEP KO OVX group, one-way ANOVA. Data are shown in each graph from left to right as 1) AEP WT sham, 2) AEP KO sham, 3) AEP WT OVX and 4) AEP KO OVX. FIG. 2, Panel C, is a set of graphs showing μCT scanning measurements of cortical bone cortical area (Ct.Ar), average cortical thickness (Ct.Th), and relative cortical bone area to tissue area (Ct.Ar/Tt.Ar). Data are shown as mean±SEM, n=5 mice per group for AEP WT sham group, n=6 mice per group for AEP KO group and n=7 mice per group for AEP WT and AEP KO OVX group, one-way ANOVA. Data are shown in each graph from left to right as 1) AEP WT sham, 2) AEP KO sham, 3) AEP WT OVX and 4) AEP KO OVX. FIG. 2, Panel D, is a set of graphs showing levels of osteocalcin, C-terminal telopeptide (CTX), serum BDNF, RANK-L, OPG, and the RANK-L/OPG ratio in wild-type and AEP knock out mice with and without ovariectomy. Data are shown as mean±SEM, left to right: n=5, 5, 7, 7 mice per group for osteocalcin and CTX measurement, n=5 mice per group for BDNF, RANK-L and OPG measurement, one-way ANOVA. Data are shown in each graph from left to right as 1) AEP WT sham, 2) AEP KO sham, 3) AEP WT OVX and 4) AEP KO OVX.

FIG. 3, Panel A, is a collection of images of hematoxylin and eosin (H&E) stained distal femur bone in wild-type (AEP WT) and AEP knockout (AEP KO) mice with (OVX) and without (Sham) ovariectomy. FIG. 3, Panel B, are images of tartrate-resistant acid phosphatase-stained (TRAP-stained) sections of the distal femur bone in AEP WT sham, AEP KO sham, AEP WT OVX and AEP KO OVX groups at low magnification (FIG. 3, Panel B, bottom images) and a selected area (shown as the boxed region in the top row of images) at higher magnification (FIG. 3, Panel B, top images). FIG. 3, Panel C, are trabecular calcein double-fluorescence labeled images of representative sections from AEP WT sham, AEP KO sham, AEP WT OVX and AEP KO OVX group. (Scale bar, 30 μm). FIG. 3, Panel D, is a set of graphs of histomorphometric indices of bone turnover (mineral apposition rate (MAR), bone formation rate per bone surface (BFR/BS), osteoblast surface per bone surface (ObS/BS), number of osteoblasts per bone surface (N.Ob/BS), number of osteoclasts per bone surface (N.Oc/BS), osteoclast surface per bone surface (OcS/BS), and mineralizing surface/bone surface (MS/BS) in AEP WT and AEP KO mice with or without ovariectomy. MAR=mineral apposition rate; BFR/BS=Bone formation rate; Ob.s/BS=percentage of bone surface covered by osteoblasts; N.Ob/BS=number of osteoblasts per mm bone surface; Oc.S/BS=percentage of bone surface covered by osteoclasts; N.Oc/BS=number of osteoclasts per mm bone surface. MS/BS=Mineralizing surface/bone surface (%). Data are shown as mean±SEM, n=6 mice per group, one-way ANOVA. Data are shown in each graph from left to right as 1) AEP WT sham, 2) AEP KO sham, 3) AEP WT OVX and 4) AEP KO OVX.

FIG. 4, Panel A are representative images of the femoral indices of trabecular bone structure measured by in vitro μCT scan in WT sham, BDNF+/−sham, WT OVX, BDNF+/−OVX, and WT OVX+R13, BDNF+/−OVX+R13 group. FIG. 4, Panel B are graphs showing the μCT scanning measurements of trabecular bone volume fraction (BV/TV), connectivity density (Conn.D), structure model index (SMI), trabecular number (Tb.N), trabecular spacing (Tb.Sp), trabecular thickness (Tb.Th). Data are shown as mean±SEM, n=8 mice per group (n=7 mice per group for BDNF+/−sham group) mice per group, one-way ANOVA. Data are shown in each graph from left to right as 1) WT sham, 2) BDNF+/−sham, 3) WT OVX, 4) BDNF+/−OVX, 5) WT OVX+R13, and 6) BDNF+/−OVX+R13. FIG. 4, Panel C are graphs showing the μCT scanning measurements of cortical bone cortical area (Ct.Ar), average cortical thickness (Ct.Th) and relative cortical bone area to tissue area (Ct.Ar/Tt.Ar). Data are shown as mean±SEM, n=8 mice per group (n=7 mice per group for BDNF+/−sham group) mice per group, one-way ANOVA. Data are shown in each graph from left to right as 1) WT sham, 2) BDNF+/−sham, 3) WT OVX, 4) BDNF+/−OVX, 5) WT OVX+R13, and 6) BDNF+/−OVX+R13. FIG. 4, Panel D are graphs showing serum levels of osteocalcin, CTX, RANK-L, OPG, RANK-L/OPG ratio and serum BDNF levels. Data are shown as mean±SEM, n=5 mice per group for BDNF measurement, n=6 mice per group for RANK-L and OPG measurement, n=7 mice per group for osteocalcin and CTX measurement, one-way ANOVA. Data are shown in each graph from left to right as 1) WT sham, 2) BDNF+/−sham, 3) WT OVX, 4) BDNF+/−OVX, 5) WT OVX+R13, and 6) BDNF+/−OVX+R13.

FIG. 5, Panel A is an in vivo pharmacokinetic (PK) study of two months old female mice treated with R13 (21.8 mg/kg) by oral gavage, where the blood and bone marrow samples were collected at different time points (0, 15, 30, 60, 120 min.) after R13 treatment. 7,8-DHF concentration in the plasma (ng/mL) and bone marrow (ng/g) samples were quantitatively analyzed by LC-MS/MS. Data are shown as mean±SEM, n=3 mice per group. FIG. 5, Panel B is a collection of Western blots showing the phosphorylation of TrkB, AKT and MAPK in bone marrow of the mice.

FIG. 6, Panel A are representative images of hematoxylin and eosin (H&E) staining of the distal femur bone in WT sham, BDNF+/−sham, WT OVX, BDNF+/−OVX, WT OVX+R13, and BDNF+/−OVX+R13 groups (n=5 mice per group). (Scale bar, 500 μm). FIG. 6, Panel B are representative images of tartrate resistant acid phosphatase-stained (TRAP-stained) sections of the distal femur bone in WT sham, BDNF+/−sham, WT OVX, BDNF+/−OVX, WT OVX+R13, and BDNF+/−OVX+R13 groups shown at low magnification (upper panel) and higher magnification (lower panel) (n=5 mice per group). (Scale bar, 500 μm (upper panels), 20 m (lower panels)). FIG. 6, Panel C are representative images of calcein double-fluorescence labeling images of the trabecular bone in WT sham, BDNF+/−sham, WT OVX, BDNF+/−OVX, WT OVX+R13, and BDNF+/−OVX+R13 groups (n=6 mice per group) (Scale bar, 30 m). FIG. 6, Panel D are graphs of histomorphometric indices of bone turnover in WT and BDNF+/−mice after OVX with or without R13 treatment. N.Oc/BS and Oc.S/BS are indices of bone resorption. N.Ob/BS, Ob.S/BS, MAR, BFR/BS and MS/BS are indices of bone formation. Data are shown as mean±SEM, n=6 mice per group, one-way ANOVA. Data are shown in each graph from left to right as 1) WT sham, 2) BDNF+/−sham, 3) WT OVX, 4) BDNF+/−OVX, 5) WT OVX+R13, and 6) BDNF+/−OVX+R13.

FIG. 7 is a collection of graphs depicting single labeled surface (sLS) measurements, double labeled surface (dLS) measurements, and inter-labeled width measurements of the femur bone. FIG. 7, Panel A are graphs of AEP wild-type (WT) or knockout (KO) mice with sham or ovariectomy. Data are shown as mean±SEM, n=6 mice per group, one-way ANOVA. Data are shown in each graph from left to right as 1) AEP WT sham, 2) AEP KO sham, 3) AEP WT OVX, and 4) AEP KO OVX. FIG. 7, Panel B are graphs of WT or BDNF+/−mice with sham or ovariectomy following R13 treatment or no R13 treatment. Data are shown as mean±SEM, n=6 mice per group, one-way ANOVA. Data are shown in each graph from left to right as 1) WT sham, 2) BDNF+/−sham, 3) WT OVX, 4) BDNF+/−OVX, 5) WT OVX+R13, and 6) BDNF+/−OVX+R13. FIG. 7, Panel C are graphs of WT with or without OVX followed by R13 or anti-RANK-L antibody treatment. Data are shown as mean±SEM, n=6 mice per group, one-way ANOVA. Data are shown in each graph from left to right as 1) Sham, 2) OVX, 3) OVX+IgG, 4) OVX+R13, and 5) OVX+anti-RANK-L.

FIG. 8, Panel A is a set of images of the femoral indices of trabecular bone structure measured by in vitro μCT scan. FIG. 8, Panel B are a collection of graphs showing that R13 increases bone volume fraction in WT mice. μCT scanning measurements of trabecular bone volume fraction (BV/TV), connectivity density (Conn.D), structure model index (SMI), trabecular number (Tb.N), trabecular spacing (Tb.Sp), trabecular thickness (Tb.Th), Cortical area (Ct.Ar) and average cortical thickness (Ct.Th). Graphs show that there is significant difference between WT and WT+R13 mice in BV/TV (p=0.0229), but no significant difference between WT and WT+R13 mice in Conn.D. (p=0.0649), SMI (p=0.2728), Tb.N (p=0.2336), Tb.Sp (p=0.2336), Tb.Th (p=0.1593), Ct.Ar (p=0.9636) and Ct.Th (p=0.3615). Data are shown as mean±SEM, n=8 mice per group, Two-tailed unpaired t-test. FIG. 8, Panel C are graphs showing serum levels of Osteocalcin, CTX, RANK-L, OPG, RANK-L/OPG ratio and serum BDNF levels. Graphs show that there is a significant difference between WT and WT+R13 mice in OPG level (p=0.0348), but no significant difference between WT and WT+R13 mice in osteocalcin (p=0.5139), CTX (p=0.2449), RANK-L (p=0.9612), RANK-L/OPG ratio (p=0.111) and BNDF (p=0.8826). Data are shown as mean SEM, n=5 mice per group for BDNF measurement, n=6 mice per group for CTX measurement or n=7 mice per group for osteocalcin, RANK-L and OPG measurement, Two-tailed unpaired t-test.

FIG. 9, Panel A contains representative images of ALP staining in MC3T3-E4 cells treated with BDNF or 7,8-DHF combined with or without K252a for 14 days (n=3 independent experiments) (Scale bar, 5 mm (upper panel), 200 μm (lower panel)). FIG. 9, Panel B contains representative images of Alizarin Red S mediated calcium staining in MC3T3-E4 cells treated with BDNF or 7,8-DHF combined with or without K252a for 21 days showed that 7,8-DHF promoted MC3T3 cells mineralization (n=3 independent experiments) (Scale bar, 5 mm (upper panel), 200 μm (lower panel)). FIG. 9, Panel C contains Western blots for MC3T3 cells cultured in complete medium or osteogenic induction medium (OIM) with BDNF or 7,8 DHF combined with or without K252a for 4 days. The Western blots show that 7,8-DHF inhibited C/EBPP/AEP pathway and increase OPG expression, and K252 inhibited the effect of 7,8-DHF. FIG. 9, Panel D contains graphs of the relative protein levels of C/EBPP, p-C/EBPβ, AEP, RANK-L, OPG, Osterix, p-TrkB/TrkB and p-Akt/AKT in MC3T3 cells cultured in the complete medium or OIM with BDNF or 7,8-DHF combined with or without K252a for four days. Data are shown as mean±SEM of three independent experiments, one-way ANOVA. Data are shown in each graph from left to right as 1) Control, 2) OIM+DMSO, 3) OIM+BDNF, 4) OIM+7,8-DHF, 5) OIM+BDNF+K252a, and 6) OIM+7,8-DHF+K252a. FIG. 9, Panel E contains graphs of an AEP enzymatic activity assay. BDNF and 7,8-DHF inhibit AEP activities, and K252a abolish BDNF and 7,8-DHF's effects. Data are shown as mean±SEM of 3 independent experiments, one-way ANOVA. Data are shown in each graph from left to right as 1) Control, 2) OIM+DMSO, 3) OIM+BDNF, 4) OIM+7,8-DHF, 5) OIM+BDNF+K252a, and 6) OIM+7,8-DHF+K252a. FIG. 9, Panel F contains graphs of qPCR results showing that OPG mRNA expression increases in MC3T3 cells after 7,8-DHF treatment for four days. Data are shown as mean±SEM of three independent experiments, one-way ANOVA. Data are shown in each graph from left to right as 1) Control, 2) OIM+DMSO, 3) OIM+BDNF, 4) OIM+7,8-DHF, 5) OIM+BDNF+K252a, and 6) OIM+7,8-DHF+K252a. FIG. 9, Panel G contains graphs showing that 7,8-DHF increases OPG and decreases the RANK-L/OPG ratio. Levels of OPG and RANK-L proteins secreted into the medium were measured by ELISA. Data are shown as mean±SEM of 3 independent experiments, one-way ANOVA. Data are shown in each graph from left to right as 1) Control, 2) OIM+DMSO, 3) OIM+BDNF, 4) OIM+7,8-DHF, 5) OIM+BDNF+K252a, and 6) OIM+7,8-DHF+K252a.

FIG. 10, Panel A is a Western blot showing the expression of AEP, p-AEP, fibronectin, osterix and RUNX2. FIG. 10, Panel B are representative images of ALP staining positive cells in MC3T3 cells cultured in OIM with or without AEP C189S plasmid, AEP WT plasmid and AEP T322E plasmid transfection for 14 days (n=3 independent experiments) (Scale bar, 5 mm (upper panel), 200 μm (lower panel)). FIG. 10, Panel C are representative images of Alizarin Red S mediated calcium staining in MC3T3-E4 cells treated with or without AEP C189S plasmid, AEP WT plasmid and AEP T322E plasmid transfection for 21 days (n=3 independent experiments) (Scale bar, 5 mm (upper panel), 200 μm (lower panel)). FIG. 10, Panel D are a collection of graphs depicting the quantification of the relative protein levels of AEP, p-AEP, fibronectin, osterix and runx2. Data are shown as mean±SEM of 3 biologically independent experiments, one-way ANOVA. Data are shown in each graph from left to right as 1) Control, 2) OIM, 3) OIM+control plasmid, 4) OIM+AEP^{C189S}, 5) OIM+AEP WT, and 6) OIM+AEP^{T322E}. FIG. 10, Panel E is a graph depicting an AEP enzymatic activity assay. Data are shown as mean±SEM of three biologically independent experiments, one-way ANOVA. Data are shown in each graph from left to right as 1) Control, 2) OIM, 3) OIM+control plasmid, 4) OIM+AEP^{C189S}, 5) OIM+AEP WT, and 6) OIM+AEP^{T322E}.

FIG. 11, Panel A are Western blots of MC3T3 cells cultured in OIM treated with 7,8-DHF in different time points. Western blotting showed that 7,8-DHF inhibited C/EBPβ, and increased AKT (S473), MAPK (p38), C-Jun, and CREB phosphorylation. FIG. 11, Panel B are graphs showing relative protein level of C/EBPβ, p-C/EBPβ, AEP, phosphorylated C-Jun, CREB, AKT, MAPK and TrkB in MC3T3 cells treated with 7,8-DHF at different time points. Data represent mean±SEM of 3 independent experiments (*P<0.05, ** P<0.01, one-way ANOVA). Data are shown in each graph from left to right at the following timepoints: 1) 0 min, 2) 5 min, 3) 10 min, 4) 15 min, 5) 30 min, and 6) 60 min. FIG. 11, Panel C are Western blots showing that knockdown of CREB blunted 7,8-DHF-induced OPG expression. FIG. 11, Panel D are graphs showing relative protein levels of RANK-L, OPG and RANK-L/OPG ratio. Data represent mean±SEM of three independent experiments (*P<0.05, ** P<0.01, one-way ANOVA). Data are shown in each graph from left to right as 1) DMSO+si-Control, 2) OIM+DMSO+si-Control, 3) OIM+7,8-DHF+si-Control, 4) OIM+7,8-DHF+si-C/EBPβ, 5) OIM+7,8-DHF+si-CREB, and 6) OIM+7,8-DHF+si-c-Jun. FIG. 11, Panel E are graphs of qPCR results showing that knockdown of CREB inhibited OPG mRNA expression induced by 7,8-DHF. Data represent mean±SEM of three independent experiments (*P<0.05, ** P<0.01, one-way ANOVA). Data are shown in each graph from left to right as 1) DMSO+si-Control, 2) OIM+DMSO+si-Control, 3) OIM+7,8-DHF+si-Control, 4) OIM+7,8-DHF+si-C/EBPβ, 5) OIM+7,8-DHF+si-CREB, and 6) OIM+7,8-DHF+si-c-Jun.

FIG. 12, Panel A is a collection of representative images of tartrate resistant acid phosphatase (TRAP) staining of RAW 264.7 cells induced by RANK-L with or without BDNF or 7,8-DHF for 4 days (n=3 independent experiments) (Scale bar, 1 mm). FIG. 12, Panel B is Western blotting showing that BDNF and 7,8-DHF inhibited C/EBPβ/AEP pathway, and activated p-TrkB and p-MAPK and p-AKT signaling. FIG. 12, Panel C is a graphic and numerical representation of the Western blotting shown in FIG. 12, Panel B. Data are shown as mean±SEM of 3 biologically independent experiments, one-way ANOVA. Data are shown in each graph from left to right as 1) DMSO, 2) DMSO+RANK-L, 3) DMSO+RANK-L+BDNF, and 4) DMSO+RANK-L+7,8-DHF.

FIG. 13, Panel A contains representative images of the femoral indices of trabecular bone structure measured by in vitro μCT scan. FIG. 13, Panel B contains μCT scanning measurements of trabecular bone volume fraction (BV/TV), Conn.D., Sstructure model index (SMI), Ttrabecular number (Tb.N), trabecular spacing (Tb.Sp), trabecular thickness (Tb.Th). Data are shown as mean±SEM, n=8 mice per group, one-way ANOVA. Data are shown in each graph from left to right as 1) Sham, 2) OVX, 3) OVX+IgG, 4) OVX+R13, and 5) OVX+anti-RANK-L. FIG. 13, Panel C contains graphs showing μCT scanning measurements of cortical bone Cortical area (Ct.Ar), average cortical thickness (Ct.Th) and relative cortical bone area to tissue area (Ct.Ar/Tt.Ar). Data are shown as mean±SEM, n=8 mice per group, one-way ANOVA. Data are shown as mean±SEM, n=8 mice per group, one-way ANOVA. Data are shown in each graph from left to right as 1) Sham, 2) OVX, 3) OVX+IgG, 4) OVX+R13, and 5) OVX+anti-RANK-L. FIG. 13, Panel D contains graphs showing serum levels of osteocalcin, CTX, RANK-L, OPG and RANK-L/OPG ratio. Data are shown as mean±SEM, n=6 mice per group, one-way ANOVA. Data are shown as mean±SEM, n=8 mice per group, one-way ANOVA. Data are shown in each graph from left to right as 1) Sham, 2) OVX, 3) OVX+IgG, 4) OVX+R13, and 5) OVX+anti-RANK-L.

FIG. 14, Panel A contains representative images of hematoxylin and eosin (H&E) staining of the distal femur bone in WT mice with sham, OVX, OVX+IgG, OVX+R13 and OVX+anti-RANK-L antibody group (n=5 mice per group). (Scale bar, 500 μm). FIG. 14, Panel B contains representative images of tartrate resistant acid phosphatase-stained (TRAP-stained) sections of the distal femur bone shown at low magnification (upper panel) and higher magnification (lower panel) (n=5 mice per group). (Scale bar, 500 μm (upper panels), 20 μm (lower panels)). FIG. 14, Panel C contains representative calcein double-fluorescence labeling images of the trabecular bone (n=6 mice per group) (Scale bar, 30 μm). FIG. 14, Panel D contains graphs of histomorphometric indices of distal femur. N.Oc/BS and Oc.S/BS are indices of bone resorption. N.Ob/BS, Ob.S/BS, MAR, BFR/BS and MS/BS are indices of bone formation. Data are shown as mean±SEM, n=6 mice per group, one-way ANOVA. Data are shown in each graph from left to right as 1) Sham, 2) OVX, 3) OVX+IgG, 4) OVX+R13, and 5) OVX+anti-RANK-L. FIG. 14, Panel E is a schematic diagram of R13 treatment on osteoporosis via elevating OPG and inhibiting AEP via activating BDNF/TrkB signaling.

### DETAILED DESCRIPTION

Provided herein are compounds and methods for their use as prodrugs to prevent and treat bone disorders. More specifically, to improve in vivo pharmacokinetic (PK) profiles, a prodrug, R13, has been prepared that releases 7,8-DHF after absorption and significantly increases its oral bioavailability. It has been reported that BDNF/TrkB signaling inhibits AEP via Akt phosphorylation of the T322 residue, suppressing AEP activation. The present disclosure shows that oral administration of R13 elicits robust TrkB receptor activation in the brain and gut and inhibits AEP via Akt-mediated T322 phosphorylation. Moreover, C/EBPβ is a pivotal transcription factor for escalating AEP expression during aging, and activation of the BDNF/TrkB pathway represses C/EBPβ/AEP signaling.

The disclosure also relates to methods of preventing or treating osteoporosis. Osteoporosis is a systemic bone disease, characterized by reduced bone mass, and disruption of normal bone architecture, resulting in bone fragility and increased risk of fractures. Bone homeostasis depends on the resorption of bone by osteoclasts and the formation of bone by osteoblasts. Osteoblasts can also affect osteoclast formation, differentiation, or apoptosis through several pathways, such as OPG/RANK-L/RANK.

### Compounds

Provided herein are compounds of Formula I:

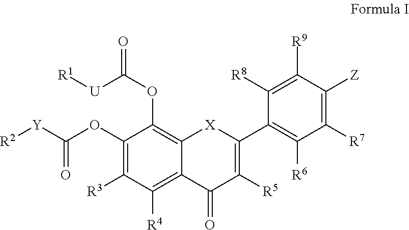

or a salt thereof, wherein:

X is O, S, or NH;

U and Y are each independently O, S, NH, N-alkyl, or CH₂;

Z is hydrogen, amino, diaminoalkyl, or heterocyclyl optionally substituted with one or more, the same or different, R¹⁵;

R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵;

R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵;

R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵;

R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and

R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl. Optionally, —O(C═O)—U—R¹ and/or —O(C═O)—Y—R² are an amino acid ester or polypeptide ester.

Optionally, X is O.

Optionally, one or both of R⁷ and R⁹ are a halogen.

Optionally, Z is hydrogen or a nonaromatic heterocyclyl bond to the phenyl ring through a nitrogen heteroatom.

Optionally, U and Y are oxygen.

Optionally, U and Y are NH or Nalkyl; and R¹ and R² are alkyl.

Optionally, R³, R⁴, R⁵, R⁶, R⁷, R⁸, and R⁹ are hydrogen.

Optionally, the compound is selected from: 4-oxo-2-phenyl-4H-chromene-7,8-diyl bis(methylcarbamate); 4-oxo-2-phenyl-4H-chromene-7,8-diyl dipropionate; 4-oxo-2-phenyl-4H-chromene-7,8-diyl bis(2,2-dimethylpropanoate); diethyl (4-oxo-2-phenyl-4H-chromene-7,8-diyl) dicarbonate; 4-oxo-2-phenyl-4H-chromene-7,8-diyl bis(ethylcarbamate); 4-oxo-2-phenyl-4H-chromene-7,8-diyl bis(dimethylcarbamate); and 4-oxo-2-phenyl-4H-chromene-7,8-diyl bis(3-methylbutanoate) or salts thereof.

As used herein a flavone refers to any compound comprising a 2-phenyl-4H-chromen-4-one ring system.

As used herein, alkyl means a noncyclic straight chain or branched, unsaturated or saturated hydrocarbon such as those containing from 1 to 10 carbon atoms, typically 1 to 6 carbon atoms. Within any embodiments, herein alkyl may refer to an alkyl with 1 to 6 carbons (C₁₋₆alkyl). Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

As used herein, non-aromatic mono or polycyclic alkyls are referred to as carbocycles or carbocylyl groups. Representative saturated carbocycles include cyclopropyl, cyclobutyl, cyclopentyl, cyclohexyl, and the like; while unsaturated carbocycles include cyclopentenyl and cyclohexenyl, and the like.

As used herein, heterocarbocycles or heterocarbocyclyl groups are carbocycles that contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

As used herein, aryl means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, heteroaryl refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term heteroaryl includes N-alkylated derivatives such as a 1-methylimidazol-5-yl substituent.

As used herein, heterocycle or heterocyclyl refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

As used herein, alkylthio refers to an alkyl group as defined above with the indicated number of carbon atoms attached through a sulfur bridge. An example of an alkylthio is methylthio, (i.e., —S—CH₃).

As used herein, alkoxy refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

As used herein, alkylamino refers an alkyl group as defined above attached through an amino bridge. An example of an alkylamino is methylamino, (i.e., —NH—CH₃). Alkyloxycarbonyl refers to an alkyl as defined above attached through a carboxy bridge (i.e., —(C═O)Oalkyl. Alkylcarbamoyl refers to an alkyl as defined above attached through a carbonyl bridge (i.e., —(C═O)NHalkyl). Alkanoyl refers to an alkyl as defined above attached through a carbonyl bridge (i.e., —(C═O)alkyl). Alkylsulfonyl refers to an alkyl as defined above attached through a sulfonyl bridge (i.e., —S(═O)₂alkyl) such as mesyl and the like, and arylsulfonyl refers to an aryl attached through a sulfonyl bridge (i.e., —S(═O)₂aryl). Alkylsulfonamide refers to an alkyl as defined above attached through a sulfamoyl bridge (i.e., —S(═O)₂NHalkyl), and an arylsulfonamide refers to an alkyl attached through a sulfamoyl bridge (i.e., (i.e., —S(═O)₂NHaryl). Alkylsulfinyl refers to an alkyl as defined attached through a sulfinyl bridge (i.e. —S(═O)alkyl).

As used herein, the terms halogen and halo refer to fluorine, chlorine, bromine, and iodine.

As used herein, the term substituted refers to a molecule wherein at least one hydrogen atom is replaced with a substituent. When substituted, one or more of the groups are substituents. The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term optionally substituted, as used herein, means that substitution is optional and therefore the designated atom can be unsubstituted.

As used herein, salts refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In a preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

As used herein, the term derivative refers to a structurally similar compound that retains sufficient functional attributes of the identified analog. The derivative may be structurally similar because it is lacking one or more atoms, is substituted, is a salt, is in different hydration/oxidation states, or one or more atoms within the molecule are switched, such as but not limited to, replacing an oxygen atom with a sulfur atom or replacing an amino group with a hydroxyl group. The derivative may be a prodrug. Derivatives may be prepared by any variety of synthetic methods or appropriate adaptations presented in synthetic or organic chemistry textbooks, such as those provided in March's Advanced Organic Chemistry: Reactions, Mechanisms, and Structure, Wiley, 8th Edition (2019), by Michael B. Smith, or Domino Reactions in Organic Synthesis, Wiley (2006), by Lutz F. Tietze, hereby incorporated by reference.

As used herein, an excipient refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

### Formulations

Pharmaceutical compositions disclosed herein may be in the form of pharmaceutically acceptable salts, as generally described below. Non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se.

When the compounds of the disclosure contain an acidic group as well as a basic group, the compounds of the disclosure may also form internal salts, and such compounds are within the scope of the disclosure. When a compound contains a hydrogen-donating heteroatom (e.g., NH), salts are contemplated to cover isomers formed by transfer of the hydrogen atom to a basic group or atom within the molecule.

Pharmaceutically acceptable salts of the compounds include the acid addition and base salts thereof. Suitable acid addition salts are formed from acids that form non-toxic salts. Examples include acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases that form non-toxic salts. Examples include aluminum, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2nd Edition 2011), incorporated herein by reference.

The compounds described herein may be administered in the form of prodrugs. A prodrug can include a covalently bonded carrier that releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include but are not limited to, acetate, formate, and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

Pharmaceutical compositions for use in the present disclosure typically comprise an effective amount of a compound and a suitable pharmaceutical acceptable carrier. The preparations may be prepared in a manner known per se, which usually involves mixing at least one compound according to the disclosure with the one or more pharmaceutically acceptable carriers, and, if desired, in combination with other pharmaceutically active compounds, when necessary under aseptic conditions. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg. Optionally, the dosages will contain between 10 and 100 mg of the at least one compound of the disclosure, e.g. about 10, 20, 30, 40, 50, 60, 70, 80, 90, or 100 mg per unit dosage.

The compounds can be administered by a variety of routes including the oral, rectal, transdermal, subcutaneous, intravenous, intramuscular, or intranasal routes, depending mainly on the specific preparation used. The compound will generally be administered in an effective amount, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the subject and the nature and severity of the disease/symptoms to be treated. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Depending upon the manner of introduction, the compounds described herein may be formulated in a variety of ways. Formulations containing one or more compounds can be prepared in various pharmaceutical forms, such as granules, tablets, capsules, suppositories, powders, controlled release formulations, suspensions, emulsions, creams, gels, ointments, salves, lotions, or aerosols and the like. These formulations are employed in solid dosage forms suitable for simple (e.g., oral) administration of precise dosages. Solid dosage forms for oral administration include, but are not limited to, tablets, soft or hard gelatin or non-gelatin capsules, and caplets. However, liquid dosage forms, such as solutions, syrups, suspension, shakes, etc. can also be utilized. Optionally, the formulation is administered topically.

Formulations containing one or more of the compounds described herein may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein carrier includes, but is not limited to, diluents, binders, lubricants, disintegrators, fillers, pH modifying agents, preservatives, antioxidants, solubility enhancers, and coating compositions.

Delayed release, extended release, and/or pulsatile release dosage formulations may be prepared as described in standard references such as “Pharmaceutical dosage form tablets,” 2nd eds. Liberman et al. (New York, Marcel Dekker, Inc., 1990); “Remington—The science and practice of pharmacy,” 23rd Edition, Academic Press, 2020; and “Pharmaceutical dosage forms and drug delivery systems,” 11th Edition, Ansel et al., (Media, Pa.: Williams and Wilkins, 2018). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as fillers, are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, and powdered sugar.

Binders are used to impart cohesive qualities to a solid dosage formulation and thus to ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glyceryl behenate, polyethylene glycol, talc, and mineral oil.

Disintegrants, used to facilitate dosage form disintegration or “breakup” after administration, generally include, but are not limited to, starch, sodium starch glycolate, sodium carboxymethyl starch, sodium carboxymethylcellulose, hydroxypropyl cellulose, pregelatinized starch, clays, cellulose, alginine, gums, or cross linked polymers, such as cross-linked PVP (Polyplasdone XL from GAF Chemical Corp).

Stabilizers are used to inhibit or retard drug decomposition reactions, which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates, and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine, and lauryl sulfobetaine.

If desired, the tablets, beads, granules, or particles may also contain a minor amount of nontoxic auxiliary substances such as wetting or emulsifying agents, dyes, pH buffering agents, or preservatives.

The concentration of the compound(s) to carrier and/or other substances may vary from about 0.5 to about 100 wt. % (weight percent). For oral use, the pharmaceutical formulation will generally contain from about 5 to about 100% by weight of the active material. For other uses, the pharmaceutical formulation will generally have from about 0.5 to about 50 wt. % of the active material.

The compositions described herein can be formulated for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington—The science and practice of pharmacy,” 23rd Edition, Academic Press, 2020. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, and hydroxyalkylcelluloses such as hydroxypropyl-cellulose, hydroxypropylmethylcellulose, sodium carboxymethylcellulose, and CARBOPOL® 934 (Lubrizol Advanced Materials, Cleveland, Ohio), polyethylene oxides and mixtures thereof. Fatty compounds include, but are not limited to, various waxes, such as carnauba wax and glyceryl tristearate; wax-type substances, including hydrogenated castor oil or hydrogenated vegetable oil; or mixtures thereof.

In certain embodiments, the plastic material is a pharmaceutically acceptable acrylic polymer, including, but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain embodiments, the acrylic polymer is comprised of one or more ammonio methacrylate copolymers. Ammonio methacrylate copolymers are well known in the art. Optionally, the polymerized copolymers consist of acrylic and methacrylic acid esters with a low content of quaternary ammonium groups.

In certain embodiments, the acrylic polymer is an acrylic resin lacquer such as that which is commercially available from Rohm Pharma under the tradename EUDRAGIT®. In certain embodiments, the acrylic polymer comprises a mixture of two acrylic resin lacquers commercially available from Rohm Pharma under the tradenames EUDRAGIT® RL30D and Eudragit® RS30D. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 can be used instead or in addition to EUDRAGIT® RL30D or EUDRAGIT® RS30D. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT® RS, and 10% EUDRAGIT® RL and 90% EUDRAGIT® RS. One skilled in the art will recognize that other acrylic polymers may also be used, such as, for example, EUDRAGIT® L.

Extended release formulations can be prepared using osmotic systems or by applying a semi-permeable coating to the dosage form. In the latter case, the desired drug release profile can be achieved by combining low permeable and high permeable coating materials in suitable proportion.

Different drug release mechanisms described herein can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system, such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolido can also be used. Polyethylene glycol, hydrophilic polymers, ethylcellulose, and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed, screened, and processed.

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, e.g., formed from acrylic acid, methacrylic acid, methyl acrylate, ethyl acrylate, methyl methacrylate and/or ethyl methacrylate, and other methacrylic resins that are commercially available under the tradename EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGIT® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein; and shellac. Combinations of different coating materials may also be used. Multi-layer coatings using different polymers may also be applied.

The coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine from clinical studies.

The coating composition may include conventional additives, such as plasticizers, pigments, colorants, stabilizing agents, glidants, etc. A plasticizer is normally present to reduce the fragility of the coating and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

The formulation can provide pulsatile delivery of the one or more inhibitors. By pulsatile is meant that a plurality of drug doses are released at spaced apart intervals of time. Generally, upon ingestion of the dosage form, release of the initial dose is substantially immediate, i.e., the first drug release pulse occurs within about one hour of ingestion. This initial pulse is followed by a first-time interval (lag time) during which very little or no drug is released from the dosage form, after which a second dose is then released. Similarly, a second nearly drug release-free interval between the second and third drug release pulses may be designed. The duration of the nearly drug-release-free time interval will vary depending upon the dosage form design e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In certain embodiments, the pulsatile release profile is achieved with dosage forms that are closed and sealed capsules housing at least two drug-containing dosage units, wherein each dosage unit within the capsule provides a different drug release profile. Control of the delayed release dosage unit(s) is accomplished by a controlled release polymer coating on the dosage unit or by incorporation of the active agent in a controlled release polymer matrix. Each dosage unit may comprise a compressed or molded tablet, wherein each tablet within the capsule provides a different drug release profile. For dosage forms mimicking a twice-a-day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form, and the third tablet releases drug at least 5 hours to approximately 18 hours following ingestion of the dosage form. It is possible that the dosage form includes more than three tablets. While the dosage form will not generally include more than a third tablet, dosage forms housing more than three tablets can be utilized.

Alternatively, each dosage unit in the capsule may comprise a plurality of drug-containing beads, granules or particles. As is known in the art, drug-containing beads refer to beads made with drug and one or more excipients or polymers. Drug-containing beads can be produced by applying drug to an inert support, e.g., inert sugar beads coated with drug or by creating a core comprising both drug and one or more excipients. As is also known, drug-containing granules and particles comprise drug particles that may or may not include one or more additional excipients or polymers. In contrast to drug-containing beads, granules and particles do not contain an inert support. Granules generally comprise drug particles and require further processing. Generally, particles are smaller than granules and are not further processed. Although beads, granules and particles may be formulated to provide immediate release, beads and granules are generally employed to provide delayed release.

In certain embodiments, the compound is formulated for topical administration. Suitable topical dosage forms include lotions, creams, ointments, and gels. A gel is a semisolid system containing a dispersion of the active agent, i.e., inhibitor, in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Methods for preparing lotions, creams, ointments, and gels are well known in the art.

The compounds described herein can be administered in combination with other active compounds. These compounds include but are not limited to alendronate sodium or alendronate sodium plus Vitamin D3 (BINOSTO® (Effrx Pharmaceuticals S.A., Freienbach, CH) or FOSAMAX PLUS D® (Merck Sharp & Dohme Corp., Whitehouse Station, N.J.)), ibandronate sodium (BONIVA® (Roche Therapeutics Inc., Nutley, N.J.)), risedronate sodium, zoledronic acid (RECLAST® (Novartis AG Corp., Basel, CH)), denosumab (PROLIA® (Amgen, Thousand Oaks, Calif.)), calcitonin-salmon, menopausal hormone therapy, raloxifene, conjugated estrogens, bazedoxifene, teriparatide (FORTEO® (Eli Lilly and Co., Indianapolis, Ind.)), abaloparatide (TYMLOS® (Radius Health, Boston, Mass.)), romosozumab-aqqg (EVENITY® (Amgen, Thousand Oaks, Calif.)).

The additional active agent(s) can be formulated for immediate release, controlled release, or combinations thereof.

### Methods of Use

Provided herein are methods of treating or preventing a disease or condition associated with bone disorders. Also described herein are methods of reducing AEP expression or promoting bone formation through the use of a prodrug or derivative thereof. The methods include administering to a subject an effective amount of one or more of the compounds, a pharmaceutically acceptable salt thereof, or pharmaceutical compositions described herein.

The compounds or pharmaceutically acceptable salts thereof and compositions described herein are useful for treating and/or preventing a disease or condition associated with bone disorders (e.g., a bone disorder associated with abnormally high bone catabolism (e.g., hyperparathyroidism), a metabolic bone disease (e.g., osteoporosis, osteomalacia, rickets, renal osteodystrophy, osteopenia, Paget's disease and osteogenesis imperfecta) or a metastatic bone disease). Optionally, in order to protect against a bone disorder, the compound, pharmaceutical salt thereof, or a composition of either may be administered prior to the development of the bone disorder or at early stages of a bone disorder. For example, the compound, pharmaceutical salt thereof, or a composition of either can be administered for treating osteopenia in a subject or treating a subject at risk of developing osteopenia or osteoporosis. Examples of a subject at risk include peri- and post-menopausal females, subjects with a history of extreme weight loss or eating disorders, subjects with hormonal disorders (e.g., overactive thyroid, parathyroid, and or adrenal glands), subjects following surgical intervention (e.g., hysterectomy or gastrointestinal surgery), subjects with a family history of a bone disorder, subjects with a history of malnutrition or malabsorption, and subjects treated with certain medications (e.g., steroids). Alternatively, the compound may be used to treat a subject diagnosed or showing symptoms of a bone disorder such as osteoporosis.

Optionally, the method of treating or preventing a bone disorder in a subject comprises administering to the subject an effective amount of a compound of Formula I or any of the compounds or compositions described above. Optionally, the compound is selected from the group consisting of 4-oxo-2-phenyl-4H-chromene-7,8-diyl bis(methylcarbamate), 4-oxo-2-phenyl-4H-chromene-7,8-diyl dipropionate, 4-oxo-2-phenyl-4H-chromene-7,8-diyl bis(2,2-dimethylpropanoate), diethyl (4-oxo-2-phenyl-4H-chromene-7,8-diyl) dicarbonate, 4-oxo-2-phenyl-4H-chromene-7,8-diyl bis(ethylcarbamate), 4-oxo-2-phenyl-4H-chromene-7,8-diyl bis(dimethylcarbamate), and 4-oxo-2-phenyl-4H-chromene-7,8-diyl bis(3-methylbutanoate) or salts thereof.

Optionally, the bone disorder comprises a bone disorder associated with abnormally high bone catabolism. Optionally, the abnormally high bone catabolism is associated with increased osteoclastogenesis in the subject, decreased osteoblastogenesis in the subject, increased osteoclast activity in the subject, decreased osteoblast activity in the subject, an imbalance of osteoclastogenesis and osteoblastogenesis in the subject, or an imbalance of osteoclast and osteoblast activity in the subject. Optionally, the bone disorder comprises a bone disorder associated with abnormally high bone catabolism (e.g., hyperparathyroidism), a metabolic bone disease (e.g., osteoporosis, osteomalacia, rickets, renal osteodystrophy, osteopenia, Paget's disease and osteogenesis imperfecta) or a metastatic bone disease.

Optionally, an additional active agent is administered, wherein the additional active agent comprises an anti-osteoporosis agent. The one or more additional agents can optionally be an antiresorptive agents (e.g., bisphosphonates), a RANK-L inhibitor, calcitonin, selective estrogen receptor modulators (SERMs)), nonsteroidal anti-inflammatory agents, or anabolic agents (e.g., sclerostin inhibitor, parathyroid hormone (PTH) analog (FORTEO®), parathyroid hormone-related protein (PTHrp) analog). Optionally, the anti-osteoporosis agent can be a monoclonal antibody. As used herein, a monoclonal antibody (e.g., a fully human or humanized antibody) or binding fragments thereof can be used as an anti-osteoporosis agent. Examples of therapeutic antibodies currently used for treating osteoporosis include Denosumab (PROLIA®) an antibody that binds to RANK-L, and Romosozumab (EVENITY®), an antibody that binds to and inhibits sclerostin. Monoclonal antibodies, such as PROLIA®, inhibit osteoclast formation by binding to the receptor activator of RANK-L, decrease bone resorption, increase bone mass density (BMD), and reduce the risk of fracture. Other monoclonal antibodies, like EVENITY® binds sclerostin, a regulatory factor in bone metabolism. Sclerostin inhibition increases bone formation and, to a lesser extent, decreases bone resorption.

Optionally, the compound, pharmaceutical salt thereof, or composition containing the compound or salt is administered orally.

Provided herein is a method of inhibiting asparagine endopeptidase (AEP) activity in a cell, comprising contacting the cell, in vivo or in vitro, with an effective amount of a compound represented by the structure of Formula I or any of the compounds, salts, or compositions described herein. Optionally, the compound is selected from the group consisting of 4-oxo-2-phenyl-4H-chromene-7,8-diyl bis(methylcarbamate), 4-oxo-2-phenyl-4H-chromene-7,8-diyl dipropionate, 4-oxo-2-phenyl-4H-chromene-7,8-diyl bis(2,2-dimethylpropanoate), diethyl (4-oxo-2-phenyl-4H-chromene-7,8-diyl) dicarbonate, 4-oxo-2-phenyl-4H-chromene-7,8-diyl bis(ethylcarbamate), 4-oxo-2-phenyl-4H-chromene-7,8-diyl bis(dimethylcarbamate), and 4-oxo-2-phenyl-4H-chromene-7,8-diyl bis(3-methylbutanoate) or salts thereof.

Optionally, the contacting increases osteoprotegerin (OPG) levels in the cell as compared to an untreated cell. As used herein, an untreated or control cell refers to a cell that has not or has not yet undergone activation or inhibition in response to a stimulus. For example, an untreated cell can be a cell that has not been contacted with a compound, salt, or composition described herein. However, such a cell can serve as a control prior to or after the effect of the contact.

Optionally, contacting a cell in vivo with a compound, salt, or composition described herein reduces trabecular bone loss, including trabecular bone loss induced by ovariectomy.

Optionally, the contacting inhibits RANK-L-induced osteoclastogenesis.

Optionally, the disclosure further provides a method of promoting bone growth, increasing bone density, or increasing bone strength in a subject, comprising administering to the subject an effective amount of a compound of Formula I or any of the compounds, salts or compositions described herein.

### Kits

Also provided herein are kits for treating or preventing bone disease in a subject. A kit can include any of the compounds, salts, or compositions described herein. For example, a kit can include a compound of Formula I, a salt thereof, or a composition containing the compound of Formula I or a salt thereof. A kit can further include one or more additional agents (e.g., an anti-osteoporosis agent) to be used in combination with any of the compounds or compositions described herein. As used herein, the term “combination with” when used to describe administration with an additional treatment means that the agents may be administered prior to, together with, or after the additional treatment, or a combination thereof. A kit can include an oral formulation of any of the compounds, salts, or compositions described herein. A kit can additionally include directions for use of the kit (e.g., instructions for treating a subject), a container, a means for administering the compounds or compositions, and/or a carrier.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs of the disease in a subject as compared to a control. As used herein, control refers to the untreated condition. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject include mammals such as humans; non-human primates, e.g., apes and monkeys; cattle; horses; sheep; rats; mice; pigs; dogs, cats, and goats. The term is not meant to be limited to a specific age or gender.

Throughout this application, various publications are referenced. The disclosures of these publications in their entireties are hereby incorporated by reference into this application.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Compound Synthesis

Substituted flavone derivatives were prepared in purity of >95%, by ¹H NMR, HPLC (HPLC, 254 nm). The synthetic route is illustrated in the schemes below. Starting materials were typically purchased from commercial sources.

### Preparation of 4-oxo-2-phenyl-4H-chromene-7,8-diyl diacetate

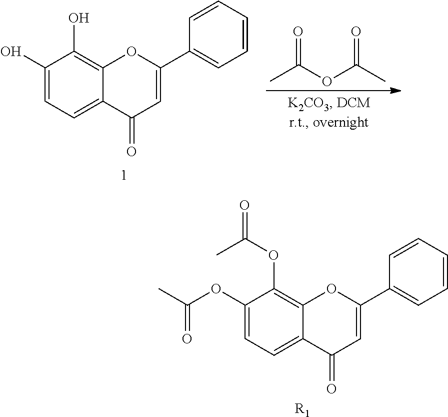

Compound 1 (100 mg, 0.4 mmol) was added to a suspension of K₂CO₃ (342 mg, 2.5 mmol) and acetic anhydride (0.1 mL, 0.8 mmol) in DCM. After stirring at r.t. overnight, the mixture was filtered and evaporated under reduced pressure. The residue was washed by ethyl ether to afford the product as a white solid (71 mg, yield: 53.3%). ¹H NMR (400 MHz, CD₃OD): δ ppm 8.06 (d, J=8.8 Hz, 1H), 7.93-7.95 (m, 2H), 7.58-7.61 (m, 3H), 7.38 (d, J=8.4 Hz, 1H), 6.93 (s, 1H), 2.48 (s, 3H), 2.37 (s, 3H). Purity: 99.8% (254 nm); MS: 339.0 [M+1]⁺

### Preparation of 4-oxo-2-phenyl-4H-chromene-7,8-diyl bis(2,2-dimethylpropanoate) compound R5

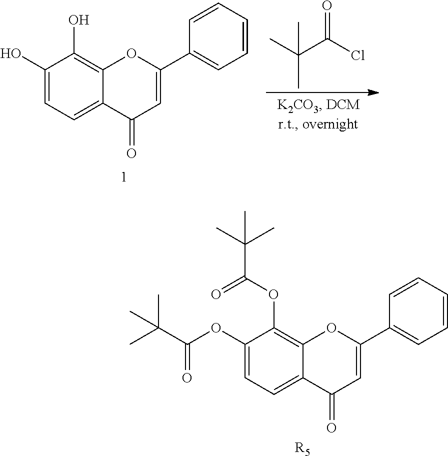

Compound 1 (150 mg, 0.6 mmol) was added to a suspension of K₂CO₃ (341 mg, 2.48 mmol) and pivaloyl chloride (0.2 mL, 1.2 mmol) in DCM. After stirring at r.t. overnight, the mixture was filtered and evaporated under reduced pressure. The residue was washed by ethyl ether to afford the product as a red solid (52 mg, yield: 20.9%). ¹H NMR (400 MHz, CDCl₃): δ ppm 8.12 (d, J=8.8 Hz, 1H), 7.80-7.82 (m, 2H), 7.50-7.55 (m, 3H), 7.20 (d, J=8.4 Hz, 1H), 6.76 (s, 1H), 1.45 (s, 9H), 1.35 (s, 9H). Purity: 99.6% (254 nm); MS: 445.1 [M+1]⁺

### Preparation of 4-oxo-2-phenyl-4H-chromene-7,8-diyl bis(3-methylbutanoate) compound R12

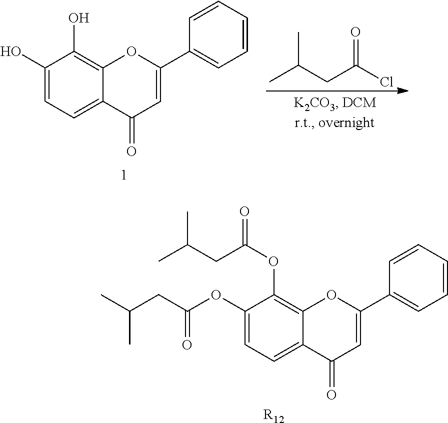

Compound 1 (150 mg, 0.6 mmol) was added to a suspension of K₂CO₃ (341 mg, 2.48 mmol) and isovaleryl chloride (0.2 mL, 1.2 mmol) in DCM. After stirring at r.t. overnight, the mixture was filtered and evaporated under reduced pressure. The residue was washed by ethyl ether to afford the product as a white solid (57 mg, yield: 23.1%). ¹H NMR (400 MHz, CDCl₃): δ ppm 8.12 (d, J=8.8 Hz, 1H), 7.80-7.82 (m, 2H), 7.48-7.55 (m, 3H), 7.23-7.26 (m, 1H), 6.78 (s, 1H), 2.59 (d, J=6.8 Hz, 2H), 2.49 (d, J=6.8 Hz, 2H), 2.24-2.32 (m, 2H), 1.09-1.11 (m, 12H). Purity: 99.6% (254 nm); MS: 445.0 [M+1]⁺

### Preparation of 4-oxo-2-phenyl-4H-chromene-7,8-diyl bis(dimethylcarbamate) compound R7

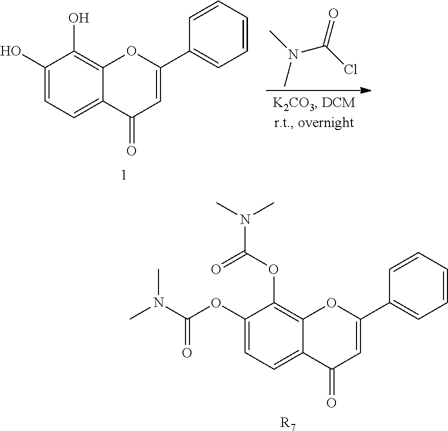

Compound 1 (200 mg, 0.8 mmol) was added to a suspension of K₂CO₃ (458 mg, 3.3 mmol) and dimethylcarbamoyl chloride (0.3 mL, 1.7 mmol) in DCM. After stirring at r.t. overnight, the mixture was filtered and evaporated under reduced pressure. The residue was washed by ethyl ether to afford the product as a white solid (53 mg, yield: 15.9%, Lot #: MC0777-38-1). ¹H NMR (400 MHz, CDCl₃): δ ppm 8.07 (d, J=8.8 Hz, 1H), 7.82-7.84 (m, 2H), 7.49-7.54 (m, 3H), 7.32-7.34 (m, 1H), 6.79 (s, 1H), 3.24 (s, 3H), 3.15 (s, 3H), 3.11 (s, 3H), 3.05 (s, 3H). Purity: 98.2% (254 nm); MS: 397.0[M+1]⁺

### Preparation of 4-oxo-2-phenyl-4H-chromene-7,8-diyl dipropionate compound R4

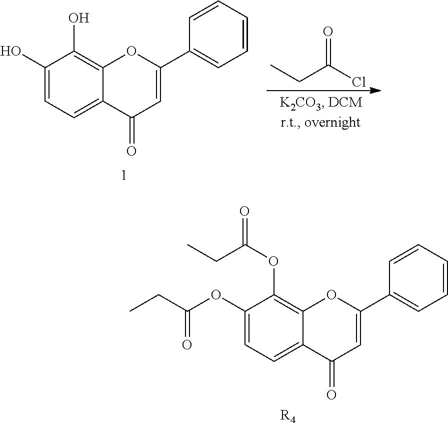

Compound 1 (200 mg, 0.8 mmol) was added to a suspension of K₂CO₃ (458 mg, 3.3 mmol) and propionyl chloride (0.3 mL, 1.7 mmol) in DCM. After stirring at r.t. overnight, the mixture was filtered and evaporated under reduced pressure. The residue was washed by ethyl ether to afford the product as a yellow solid (51 mg, yield: 17.4%). ¹H NMR (400 MHz, CDCl₃): δ ppm 8.13 (d, J=8.8 Hz, 1H), 7.79-7.81 (m, 2H), 7.51-7.55 (m, 3H), 7.25-7.27 (m, 1H), 6.79 (s, 1H), 2.75 (q, J=7.6 Hz, 2H), 2.65 (q, J=7.6 Hz, 2H), 1.38 (t, J=7.6 Hz, 3H), 1.31 (t, J=7.6 Hz, 3H). Purity: 95.5% (254 nm); MS: 367.0 [M+1]⁺

### Preparation of diethyl (4-oxo-2-phenyl-4H-chromene-7,8-diyl) dicarbonate compound R3

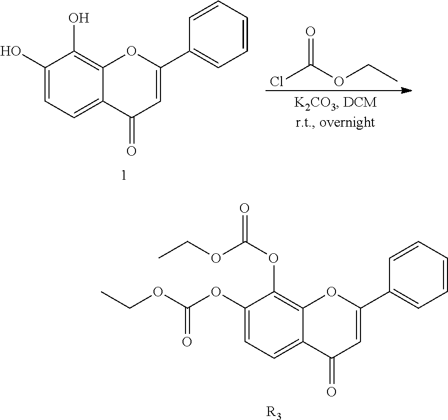

Compound 1 (200 mg, 0.8 mmol) was added to a suspension of K₂CO₃ (458 mg, 3.3 mmol) and ethyl chloroformate (0.3 mL, 1.7 mmol) in DCM. After stirring at r.t. overnight, the mixture was filtered and evaporated under reduced pressure. The residue was washed by ethyl ether to afford the product as a white solid (50 mg, yield: 15.9%). ¹H NMR (400 MHz, CDCl₃): δ ppm 8.14 (d, J=8.8 Hz, 1H), 7.85-7.87 (m, 2H), 7.50-7.56 (m, 3H), 7.35-7.38 (m, 1H), 6.83 (s, 1H), 4.35-4.43 (m, 4H), 1.41-1.44 (m, 6H). Purity: 96.0% (254 nm); MS: 399.0 [M+1]⁺

### Preparation of 4-oxo-2-phenyl-4H-chromene-7,8-diyl bis(ethylcarbamate) compound R6

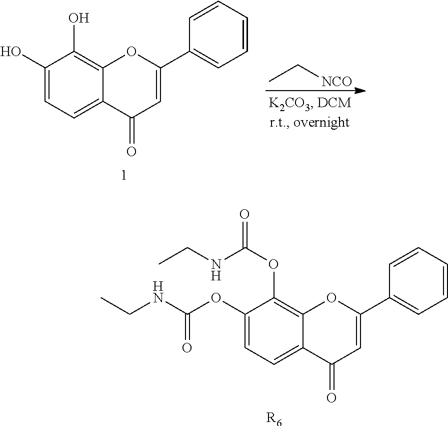

Compound 1 (200 mg, 0.8 mmol) was added to a suspension of K₂CO₃ (458 mg, 3.3 mmol) and ethyl isocyanate (0.3 mL, 1.7 mmol) in DCM. After stirring at r.t. overnight, the mixture was filtered and evaporated under reduced pressure. The residue was washed by ethyl ether to afford the product as a white solid (70 mg, yield: 22.5%). ¹H NMR (400 MHz, CDCl₃): δ ppm 8.05 (d, J=8.8 Hz, 1H), 7.84-7.86 (m, 2H), 7.47-7.53 (m, 3H), 7.28-7.30 (m, 1H), 6.79 (s, 1H), 5.45-5.46 (m, 1H), 5.22-5.23 (m, 1H), 3.32-3.54 (m, 4H), 1.23-1.30 (m, 6H). Purity: 99.8% (254 nm); MS: 397.1 [M+1]⁺.

### Preparation of 4-oxo-2-phenyl-4H-chromene-7,8-diyl bis(methylcarbamate) compound R13

Compound R13 was prepared using conditions similar to those used to prepare compound R₆, as detailed above. 1H NMR (300 Mhz, d-DMSO): δ ppm 8.21 (d, J=4.50 Hz, 1H), 7.95-8.02 (m, 3H), 7.90 (d, J=9.0 Hz, 1H), 7.58-7.64 (m, 3H), 7.38 (d, J=8.70 Hz, 1H), 7.10 (s, 1H), 2.77 (d, J=4.80 Hz, 3H), 2.69 (d, J=4.80 Hz, 3H). Purity: 99.9% (254 nm); MS: 369.1 [M+1]⁺.

### Example 2: Knockout of AEP Improves Trabecular Bone Density in Ovariectomized Female Mice

### Sample Preparation Methods

Animals

Female C57BL6/J wild-type mice and BDNF+/−mice were obtained from Jackson Laboratory (MMRRC stock #000664 and 002267), then held and bred. The AEP knockout mice were generated on a mixed C57BL/6 and 129/Ola background. All in vivo experiments were carried out in female mice. All mice were kept under specific pathogen-free conditions in an environmentally controlled clean room and housed at 22° C. on a 12-h/12-h light/dark cycle. Food and water were provided ad lib. WT, BDNF+/−mice, AEP WT and AEP knockout mice were bilaterally ovariectomized or sham operated at 12 weeks of age. One week after ovariectomy, the WT and BDNF+/−mice received vehicle or R13 dissolved in 5% DMSO/0.5% methylcellulose at dose of 21.8 mg/kg/d, six days per week, for 8 weeks by gavage. In another group, 4 weeks after ovariectomy, WT mice were treated with IgG or anti-RANK-L monoclonal antibody (10 mg/kg, twice per week) by single subcutaneous injection for 4 weeks.

Cell Culture

Murine MC3T3-E1 (subclone 4) cells and RAW 264.7 cells were obtained from American Type Culture Collection (ATCC, Manassas, Va., USA, catalog #: CRL-2593 and catalog #: TIB-71). The MC3T3-E1 cells were cultured in α-MEM (Gibco, cat. A1049001) with 10% FBS and 0.1% penicillin/streptomycin, but without ascorbic acid. The RAW 264.7 cells were cultured in DMEM supplemented with 10% FBS and 0.1% penicillin-streptomycin. The cells were maintained at 37° C. in a humidified atmosphere of 95% air and 5% CO₂.

Antibody and Reagents

Antibodies to C/EBPβ (HT-7) (catalog #: sc-7962), RANK-L (catalog #: sc-377079), OPG (catalog #: sc-390518), osterix (catalog #: sc-393325) and RUNX2 (catalog #: sc-101145) were obtained from Santa Cruz Biotechnology (Dallas, Tex.); antibodies to Legumain (D6S4H) (catalog #: 93627), p-C/EBPβ (catalog #: 3084s), AKT (catalog #: 4691s), p-AKTS473 (catalog #: 9271s), MAPK (catalog #: 9102s), p-MAPK (catalog #: 9106s), p-c-Jun (Ser73) (catalog #: 3270T), c-Jun (catalog #: 9165T), CREB (catalog #: 9197T) and p-CREB (catalog #: 9198T) were purchased from Cell Signaling Technology (Danver, Mass.); antibody to TrkB (catalog #: MAB397) was from R&D Systems (Minneapolis, Minn.); antibody to 3-actin (catalog #: A5316) were from Sigma-Aldrich (St. Louis, Mo.); antibody to fibronectin was from Millipore Sigma (Burlington, Mass.); antibodies to p-AEP (T322) and p-TrkB (Tyr816), anti-RANK-L monoclonal antibody (catalog #: 510012)m and anti-IgG antibody (catalog #: 401412) for mice treatment was obtained from Ichorbio (Oxfordshire, UK). Alpha-MEM (catalog #: A1049001) was obtained from Gibco (Waltham, Mass.). Lipo3000 transfection reagent (catalog #: L3000008) was obtained from Invitrogen (Waltham, Mass.). K252a (catalog #: ab120419) was obtained from Abcam (Cambridge, UK). TRACP&ALP double-staining kit (catalog #: MK300) was from TakaRa Bio (Kusatsu, Shiga, JP). Alkaline Phosphatase Assay kit (catalog #: ab83369) was from Abcam (Cambridge, UK). The AEP substrate Z-Ala-Ala-Asn-AMC (catalog #: 4033201) was from Bachem (Bubendorf, CH), and EZ-Link Sulfo-NHS-LC-Biotinylation Kit (catalog #: 21435) was obtained from Thermo Fisher (Waltham, Mass.). Serum Osteocalcin elisa kit (catalog #: NBP2-68151) was from Novus Biologicals (Littleton, Colo.), CTX elisa kit (catalog #: AC-06F1) was from Immunodiagnostic Systems (East Boldon, UK), RANK-L (catalog #: ab269553) and OPG (catalog #: ab203365) was from Abcam (Cambridge, UK). All chemicals not mentioned above were purchased from Sigma-Aldrich (St. Louis, Mo.).

Osteogenic Differentiation

MC3T3-E1 cells were seeded onto plates in complete medium and cultured for 24 days, until the cells reached 70% confluence. To initiate the differentiation, the cells were incubated in osteogenic induction medium (OIM) containing α-MEM, 10% FBS, dexamethasone (10-7M), (3-glycerophosphate (10 mM), and ascorbic acid (50 μg/ml). The differentiation medium was replaced every 3 days, with DMSO, BDNF (50 ng/ml) or 7,8 DHF (0.5 μM) added into the medium with or without the pretreatment of K252a (100 nM). The MC3T3-E1 cells were transfected with AEPC189S, AEP WT, AEP T322E plasmid, C/EBPβ siRNA (sc-29862, Santa Cruz Biotechnology, Dallas, Tex.), CREB siRNA (sc-35111, Santa Cruz Biotechnology, Dallas, USA), C-Jun siRNA (sc-29224, Santa Cruz Biotechnology, Dallas, Tex.) or control plasmid or control-siRNA (sc-44237, Santa Cruz Biotechnology, Dallas, Tex.) by Lipo3000 transfection reagent (Invitrogen, Waltham, Mass.) according to the instructions.

Osteoclast Differentiation

RAW264.7 cells were seeded in 24 wells plates and cultured for 24 hours in DMEM with 10% FBS and 0.1 penicillin/streptomycin. The medium was changed to α-MEM with 5% FBS, 0.1% penicillin/streptomycin. The receptor activator of NF-κB ligand (RANK-L, 30 ng/ml) was added to induce osteoclast differentiation. The medium was replaced every 3 days, accompanied with DMSO, BDNF (50 ng/ml) or 7,8 DHF (0.5 μM) added into the medium.

ALP Staining

MC3T3-E1 cells were plated in 24-well plates, cultured in complete medium or OIM, and treated with BNDF (50 ng/ml) or 7,8 DHF (0.5 μM) with or without the pretreatment of K252a (100 nM) for 14 days. The Cells were washed in PBS twice, fixed for 10 minutes with fixing buffer at room temperature, and stained with ALP staining with the TRACP&ALP double-staining kit (TaKaRa, Japan, Cat. #MK300).

Alizarin Red S Staining

MC3T3-E1 cells were plated in 24-well plates, cultured in complete medium of OIM, and treated with BDNF (50 ng/ml) or 7,8 DHF (0.5 μM) with or without pretreatment of K252a (100 nM), and then were washed in distilled water twice, and fixed in 70% ice-cold ethanol. Then the cells were stained with 2% Alizarin Red S solution (Sigma, St. Louis, Mo., USA) to detect calcification.

TRAP Staining

RAW 264.7 cells were cultured in α-MEM with or without RANK-L, in the presence or absence of BNDF (50 ng/ml) or 7,8 DHF (0.5 μM) for 5 days. The cells were washed in PBS twice, fixed in fixing solution for 10 minutes at room temperature, and then stained the TRAP activity with the TRACP&ALP double-staining kit (TaKaRa, Japan, Cat. #MK300).

ALP Activity Assay

MC3T3-E1 cells were incubated in osteogenic induction medium (OIM), followed with the treatment of different doses of 7,8-DHF (0, 5, 10, 50, 100, 500, 1000 ng/ml) for 14 days. The cells were washed with cold PBS and harvested in assay buffer. The ALP enzyme activities were analyzed by an alkaline phosphatase assay kit.

In Vivo PK of 7,8-DHF

Two month old female mice were treated with oral administration of R13 (21.8 mg/kg), and then were sacrificed to collect the serum and bone marrow at different time points (0, 15, 30, 60, 120 min) after oral gavage of R13 with 3 mice/group. Concentrations of 7,8 DHF in the plasma and bone marrow samples were determined by LC-MS/MS.

Western Blotting

MC3T3-E1 and RAW 264.7 cells were washed with ice-cold PBS and lysed in (50 mM Tris, pH 7.4, 40 mM NaCl, 1 mM EDTA, 0.5% Triton X-100, 1.5 mM Na₃VO₄, 50 mM NaF, 10 mM sodium pyrophosphate, 10 mM sodium β-glycerophosphate, supplemented with protease inhibitor cocktail) at 4° C. for 0.5 h, and centrifuged for 25 min at 15,000 rpm. The supernatant was boiled in SDS loading buffer. After SDS-PAGE, the samples were transferred to a nitrocellulose membrane. The membrane was blocked with TBS containing 5% nonfat milk and 0.1% Tween 20 (TBST) at room temperature for 2 hours, followed by the incubation with primary antibody at 4° C. overnight, and with the secondary antibody at room temperature for 2 hours. After washing with TBST, the membrane was developed using the enhanced chemiluminescent (ECL) detection system.

AEP Activity Assay

Cell lysates (10 μg) were incubated in 200 μl assay buffer (20 mM citric acid, 60 mM Na₂HPO₄, 1 mM EDTA, 0.1% CHAPS, and 1 mM DTT, pH 6.0) containing 20 μM δ-secretase substrate Z-Ala-Ala-Asn-AMC (Bachem, Bubendorf, Switzerland). AMC released by substrate cleavage was quantified by measuring at 460 nm in a fluorescence plate reader at 37° C. for 2 h in kinetic mode.

Quantitative Real-Time PCR Analysis

Total RNA was isolated by TRIzol (Life Technologies, Carlsbad, Calif.). Reverse transcription was performed with SuperScript III reverse transcriptase (Life Technologies). Gene-specific primers and probes were designed and bought from Taqman (Life Technologies). All real-time PCR reactions were performed using the ABI 7500-Fast Real-Time PCR System and the Taqman Universal Master Mix Kit (Life Technologies). The relative quantification of gene expression was calculated using the ΔΔCt method. Predesigned real-time PCR primers from Applied Biosystems (Waltham, Mass.) were used for the analysis of Opg (Tnfrsf11b; Mm0043545_m1), RANK-L (Tnfsf11; Mm00441908_m1), AEP (Lgmn; Mm01325350_m1), GAPDH (Gapdh; Mm99999915_g1).

μCT Measurements

μCT scan and analysis was performed in femurs ex vivo using a μCT-40 scanner. Voxel sizes were 12 μm³ for the in vitro measurements of femurs. For the femoral trabecular region, 140 slices were analyzed, beginning 50 slices below the distal growth plate. X-ray tube potential was 70 kVp, and integration time was 200 ms. Representative samples were reconstructed in 3D to generate visual representations of trabecular structure.

Quantitative Bone Histomorphometry

The measurements, indices and units for histomorphometric analysis were recommended by the Nomenclature Committee of the American Society of Bone and Mineral Research. Mice were injected with calcein (25 μg/g) subcutaneously at day 10 and day 3 before sacrifice. Bone histomorphometric analysis was performed at the University of Alabama at Birmingham Center for Metabolic Bone Disease-Histomorphometry and Molecular Analysis Core Laboratory. The Goldner's trichrome-stained plastic-embedded sections of calcein-double labeled femora of the mice were analyzed by an operator blinded as to the nature of the samples.

Data are presented as mean±SEM. Statistical significance was determined by a one-way ANOVA test. Values of *P<0.05 and ** P<0.01 were considered for significance.

Biochemical Markers of Bone Turnover

Serum Osteocalcin (Novus Biologicals, Littleton, Colo., Cat. NBP2-68151), CTX (Immunodiagnostic systems, East Boldon, UK, Cat. AC-06F1), RANK-L and OPG (Abcam, Cat. ab269553 and ab203365) were measured by specific Elisa assays.

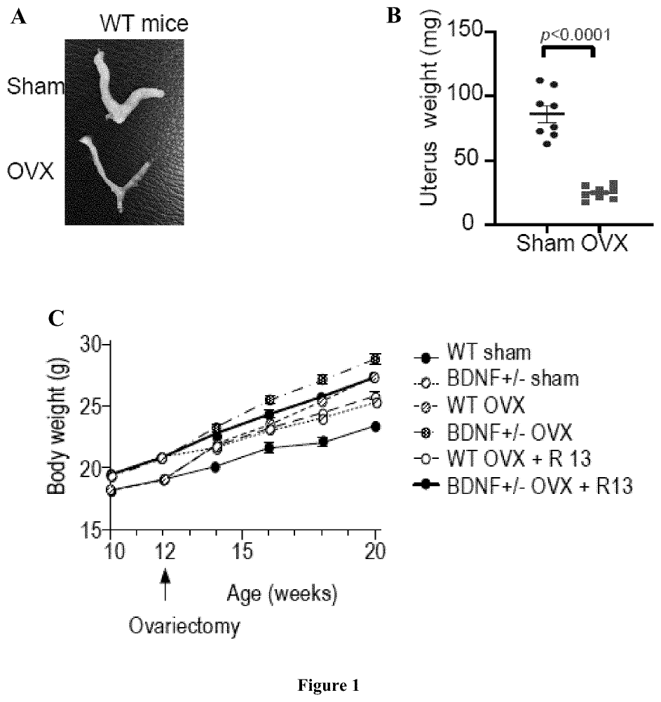

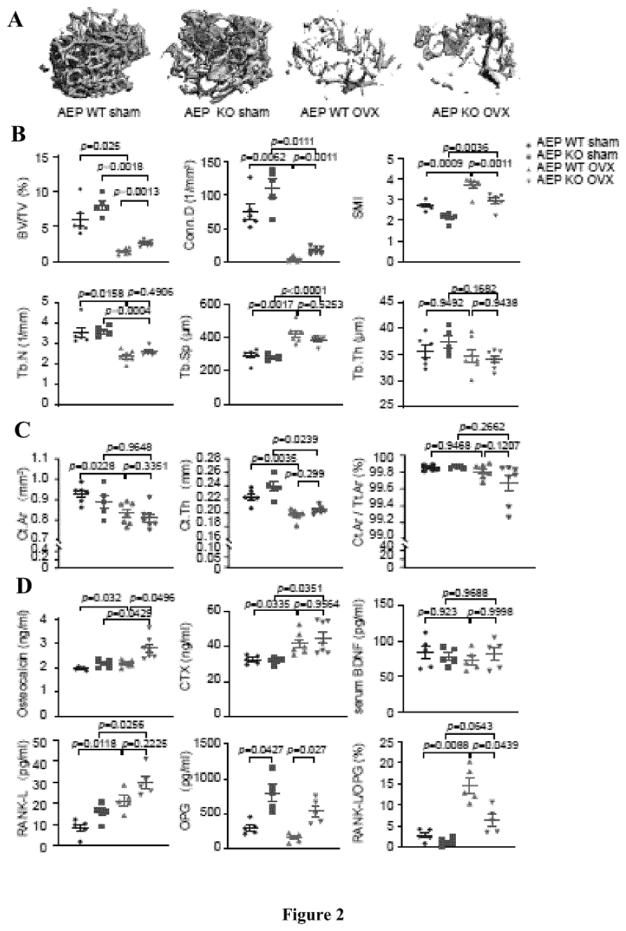

To explore the role of AEP in bone remodeling, AEP knockout mice (AEP KO) and WT littermates were subjected to ovariectomy (OVX) at the age of 12 weeks. As expected, the shrunken uterine morphology and reduced uterus weight revealed that OVX surgery was successful (FIG. 1). Microcomputed tomography (μCT) analysis of femurs harvested at sacrifice revealed a higher trabecular bone volume fraction (BV/TV), Conn.D and a lower Structure model index (SMI) in AEP KO mice compared with AEP WT mice after OVX. Moreover, OVX decreased trabecular number (Tb.N) and increased trabecular separation (Tb.Sp), while trabecular thickness (Tb.Th) indices were similar among the groups. These indices remained similar between two types of mice under sham operation (FIG. 2, Panel A and FIG. 2, Panel B). μCT scanning demonstrated that cortical bone Cortical area (Ct.Ar) and average cortical thickness (Ct.Th) were reduced upon OVX in both WT and AEP KO mice; however, relative cortical bone area to tissue area (Ct.Ar/Tt.Ar) remained comparable among the groups (FIG. 2, Panel C). Notably, levels of serum osteocalcin, a marker of bone formation, were increased after OVX, with AEP knockout (KO) significantly higher than wild-type (WT). Serum BDNF was comparable among the 4 groups. Quantification of bone resorption indices in the serum showed that the concentrations of C-terminal telopeptide of collagen (CTX), a marker for bone resorption, and RANK-L were increased after OVX. Moreover, OPG concentrations were much higher in AEP KO mice than WT mice under both OVX and sham conditions, suggesting that AEP antagonizes OPG expression under the physiological condition. Consequently, the ratios of RANK-L/OPG were substantially higher in OVX groups than sham groups with AEP KO mice lower than WT mice, in alignment with higher bone density in AEP KO group versus WT group after OVX (FIG. 2, Panel D). Hence, AEP deletion diminishes the ratio of RANK-L/OPG, leading to increased trabecular bone density after OVX.

### Example 3: Deletion of AEP Inhibits the Bone Turnover Induced by Ovariectomy

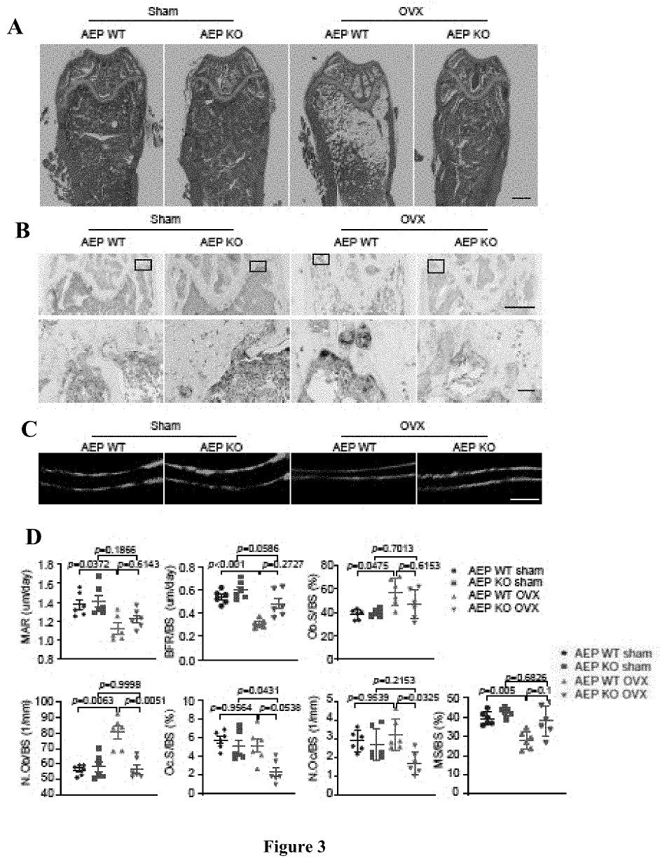

To further characterize the roles of AEP in OVX-induced osteoporosis, the H&E staining was performed and the bone morphology and white adipocytes in both animals after OVX surgery were analyzed. White adipocytes were reduced and trabecular bone was increased in the bone from AEP KO mice after OVX as compared to WT mice (FIG. 3, Panel A). Tartrate-resistant acid phosphatase (TRAP) staining revealed that OVX induced more osteoclast cells in WT than AEP KO mice (FIG. 3, Panel B). Calcein double-fluorescence labeling allows the determination of the onset time and location of mineralization and the direction and speed of bone formation. Based on dynamic indices of femur trabecular bone formation, no significant difference in mineral apposition rate (MAR) and bone formation rate (BFR) was found between WT and AEP KO sham mice, but OVX significantly decreased the MAR and BFR in WT mice, as AEP knockout alleviated this difference (FIG. 3, Panel D). Representative data of double labeling in trabecular bone are shown in FIG. 3, Panel C. Analysis of static indices of bone formation and resorption revealed that both the number of osteoclasts (N. Oc/BS) and the percentage of surfaces covered by osteoclasts (OcS/BS) were greatly decreased in AEP KO mice compared with WT mice after OVX. On the other hand, OVX also elicited a compensatory increase of number of osteoblasts (N. Ob/BS) in AEP WT group but not in AEP KO mice. Although the MS/BS (Minirelizing Surface/Bone Surface) ratios were significantly reduced in WT mice after OVX, they were unchanged in AEP KO mice (FIG. 3, Panel D). Together, these data suggest that AEP deficient mice exhibit a higher bone formation and a lower bone resorption after OVX.

### Example 4: R13 Increases OPG Levels and Blocks Trabecular Bone Loss Induced by Ovariectomy

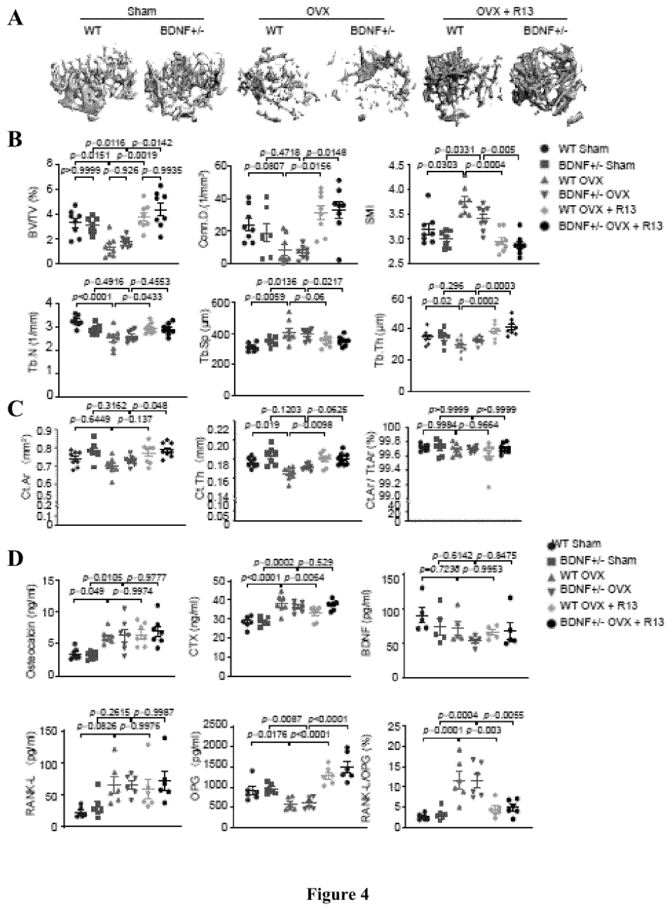

To explore the biological roles of BDNF/TrkB signaling in OVX induced bone loss, 12 week old female BDNF+/−mice and WT littermates were studied. Four days after OVX surgery, WT and BDNF+/−mice were administered either R13 (21.8 mg/kg) or vehicle, orally, six days per week for eight weeks. Assessment of femoral bone structure by in vitro μCT revealed that trabecular bone density, expressed as a function of total tissue volume fraction (BV/TV), was dramatically decreased, and structure model index (SMI) was increased by OVX in both WT and BDNF+/−mice. However, R13 treated-OVX mice showed a higher BV/TV, Conn.D and a lower SMI compared to OVX group. Quantification of parameters of trabecular structure revealed that R13-treated OVX mice displayed higher trabecular thickness (Tb.Th) and trabecular number (Tb.N) than vehicle control, decreased trabecular spacing (Tb.Sp) in both type of mice as compared with the OVX-treated group (FIG. 4, Panel A and FIG. 4, Panel B). μCT scanning showed that cortical bone cortical area (Ct.Ar) and average cortical thickness (Ct.Th) were escalated by R13 after OVX in both WT and BDNF+/−mice; nonetheless, the relative cortical bone area to tissue area (Ct.Ar/Tt.Ar) ratios remained unchanged among the groups (FIG. 4, Panel C). Assessments of the serum levels of CTX and osteocalcin indicated osteocalcin was increased in R13-treated OVX mice compared with the vehicle-treated sham group, and OVX-treated and R13-treated OVX mice exhibited higher CTX levels compared to the sham group. Nevertheless, OPG were substantially increased upon R13 treatment, leading to significant reduction in RANK-L/OPG ratios in both WT and BDNF+/−mice, though the serum BDNF levels remained equivalent among the groups (FIG. 4, Panel D). Hence, BDNF haploinsufficiency does not alter femur trabecular bone properties after OVX, but treatments with R13 strongly increase bone density.

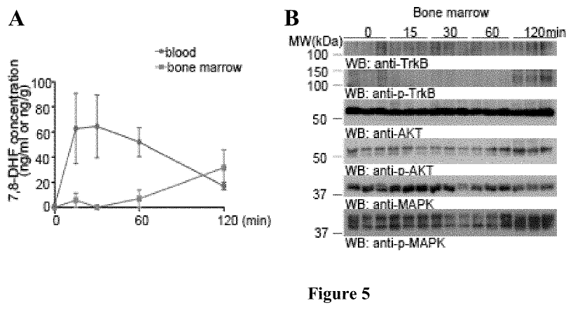

To explore whether orally administered R13 released sufficient 7,8-DHF in the bone marrow to trigger osteoblast differentiation, an in vivo PK (pharmacokinetics) study was conducted. 7,8-DHF distribution was found to be time-dependently increased in the bone marrow, reaching 31.7 ng/mL (˜125 nM), which is much higher than the EC₅₀ (˜50 nM) for activating TrkB in primary neurons. Its concentration started to increase in the plasma after oral gavage and climaxed around 15-30 min and declined since then, and reached 17.1 ng/ml at 120 min (FIG. 5, Panel A). Immunoblotting of p-TrkB signaling in the bone marrow showed that 7,8-DHF concentrations correlated with neurotrophic signaling activation (FIG. 5, Panel B). Thus, R13-derived 7,8-DHF activates BDNF/TrkB signaling in the bone marrow after oral administration of R13.

### Example 5: R13 Blocks the Changes in Bone Turnover Induced by Ovariectomy

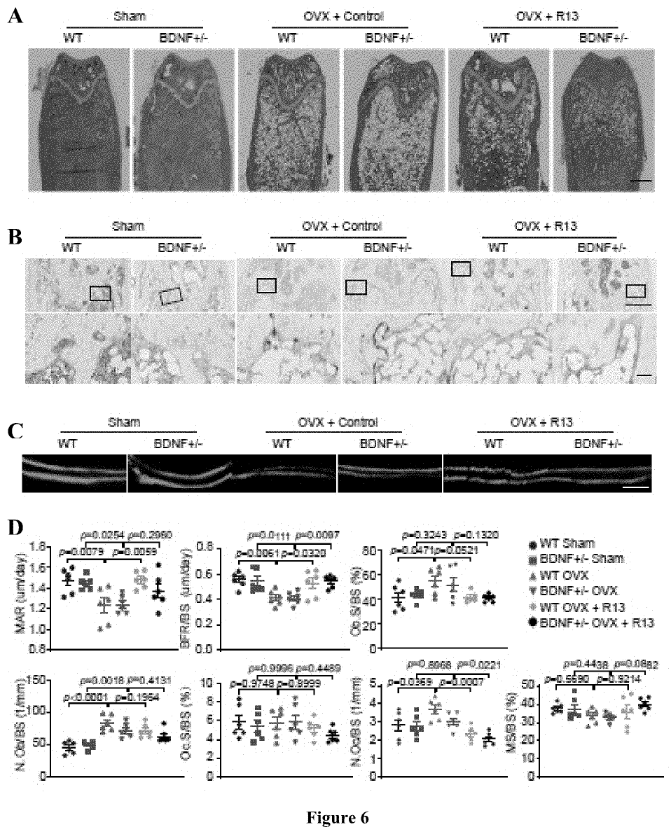

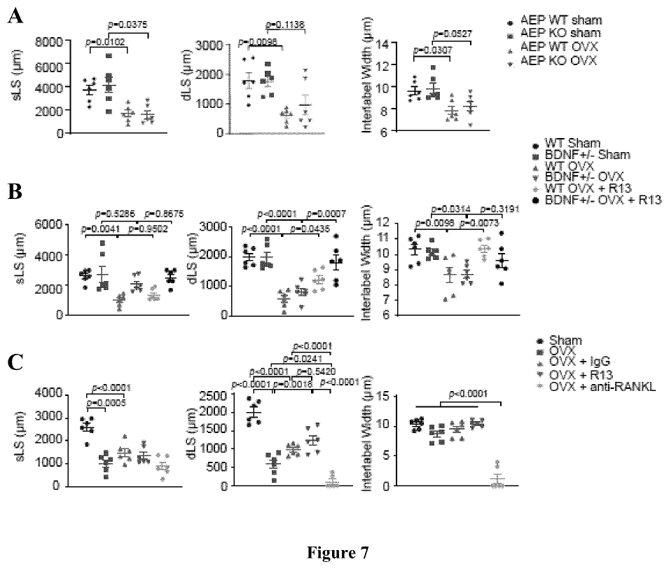

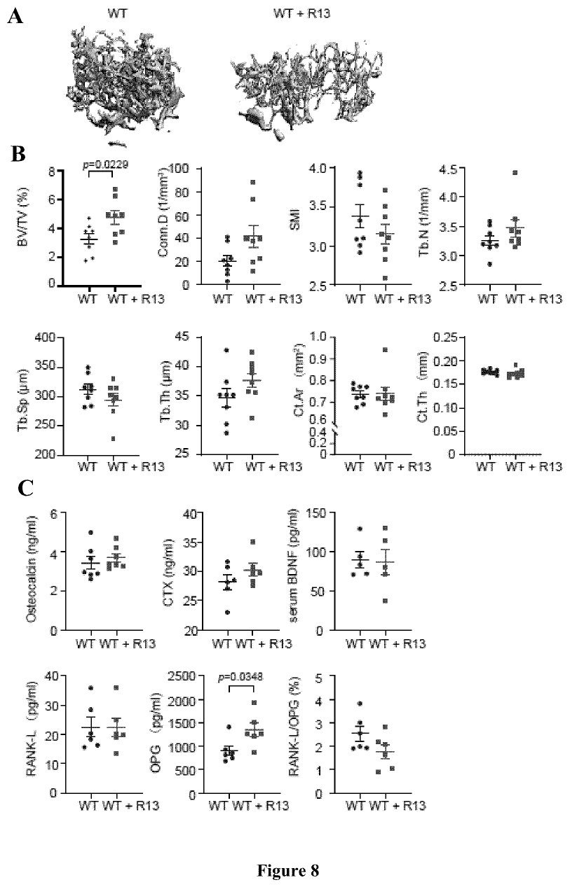

To further explore the roles of BDNF signaling in bone resorption and formation after OVX, hematoxylin and eosin (H&E) staining was conducted to analyze bone morphology and white edipocytes in both WT and BDNF+/−mice after OVX surgery. R13 treatment increased trabecular bone tissue and decreased the adipocyte content in the bone after OVX (FIG. 6, Panel A). TRAP staining revealed that OVX-induced demonstrable osteoclast cells in both WT and BDNF+/−mice were diminished by R13 treatments (FIG. 6, Panel B). Dynamic indices of bone formation showed that vehicle-treated mice exhibited lower MAR and BFR as compared to R13-treated mice after OVX. Representative data of double labeling in trabecular bone are shown in FIG. 6, Panel C, indicating that OVX-induced bone loss is attenuated by R13 treatment via an increase in bone formation. These observations were also validated by the single and double-labeled surface and inter-label thickness analysis (FIG. 7). By contrast, no significant differences in ObS/BS and N.Ob/BS, which are static indices of bone formation, were found in vehicle and R13-treated mice. Treatments with R13 inhibited N.Oc/BS in both WT and BDNF+/−mice after OVX. Nonetheless, the percentage of surfaces covered by OCs (Oc.S/BS) and MS/BS ratios remained comparable among the groups regardless of the treatment (FIG. 6, Panel D). Hence, R13 treatment appears to induce bone formation and inhibit bone resorption after OVX. Remarkably, R13 significantly increased OPG levels without affecting RANK-L and it also elevated BV/TV ratio in WT mice without any surgery (FIG. 8). Thus, R13 treatment greatly blocks the bone loss induced by OVX and substantially elevates OPG levels.

### Example 6: 7,8-DHF Promotes MC3T3-E4 Cell Differentiation, Mineralization and OPG Secretion

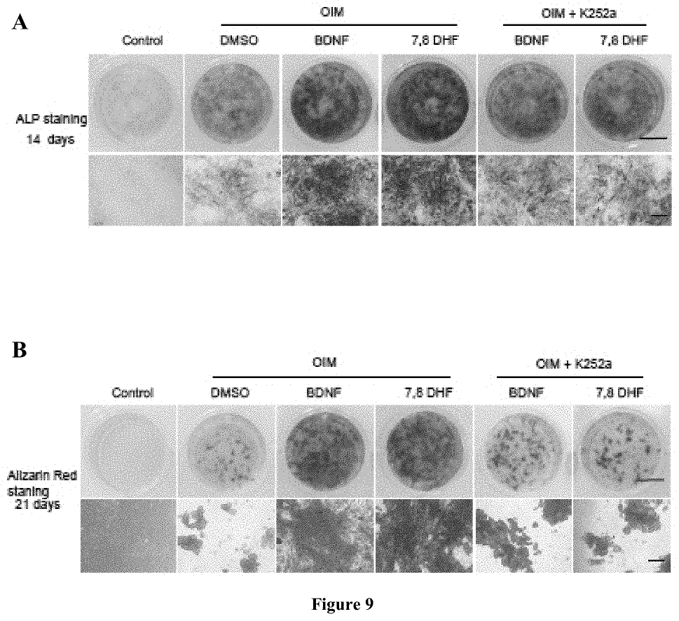

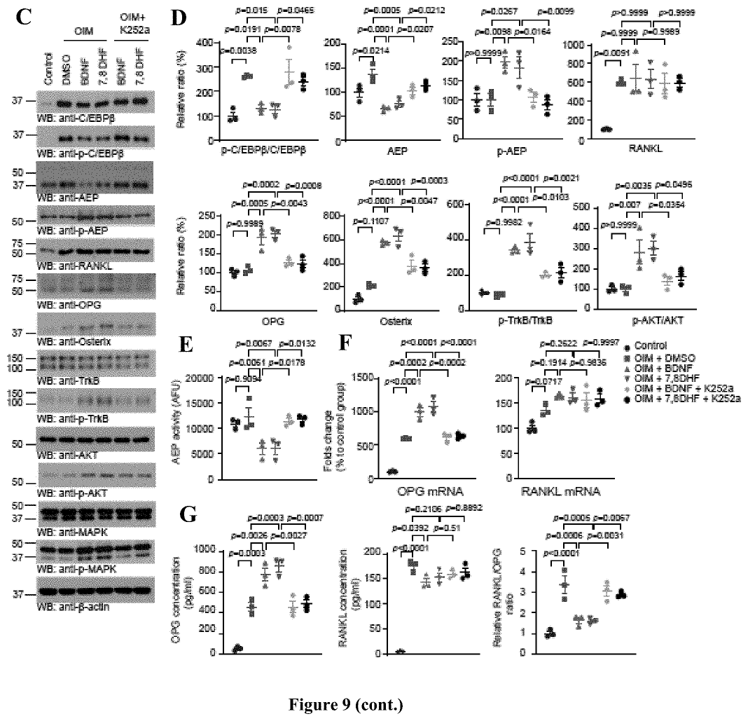

7,8-DHF binds to TrkB receptor extracellular region, whereas BDNF interacts with TrkB receptors, mimicking the biological actions of BDNF in a TrkB-dependent manner. To examine the molecular mechanisms of how 7,8-DHF stimulates bone density elevation, the effects of 7,8-DHF were tested on MC3T3-E1 cells in the presence of OIM (osteogenic induction medium). Alkaline phosphatase (ALP) staining showed that OIM treatment at 14 days enhanced osteoblast cell differentiation, which was further escalated by BDNF or 7,8-DHF, respectively. Alizarin Red staining also validated these observations at 21 days (FIG. 9, Panel A and FIG. 9, Panel B), supporting the conclusion that BDNF or 7,8-DHF strongly stimulates MC3T3-E1 differentiation and calcium deposition.

Stimulation of the BDNF/TrkB pathway inhibits AEP activation via Akt phosphorylation of T322 residue, sequestering AEP into lysosomes. Stimulation of the BDNF/TrkB pathway decreases AEP expression levels via repressing its transcription factor C/EBPβ. Immunoblotting revealed that OIM robustly induced p-C/EBPβ and total C/EBPβ expression in MC3T3-E1 cells, both of which were distinctly repressed by either BDNF or 7,8-DHF. Consequently, expression of the downstream effector, AEP, was diminished, which inversely resulted in RANK-L and OPG augmentation. Osterix, a key early gene in the bone formation cascade, is usually used as a predictive measure of bone formation. As expected, OIM prominently elevated Osterix levels as compared with vehicle. Similar findings occurred in the presence of BDNF or 7,8-DHF. Consequently, these effects triggered by BDNF or 7,8-DHF were potently blunted by Trk receptors inhibitor K252a (FIG. 9, Panel C and FIG. 9, Panel D). In alignment with active AEP repression by BDNF or 7,8-DHF, the enzymatic assay validated that AEP protease activities were greatly blocked (FIG. 9, Panel E).

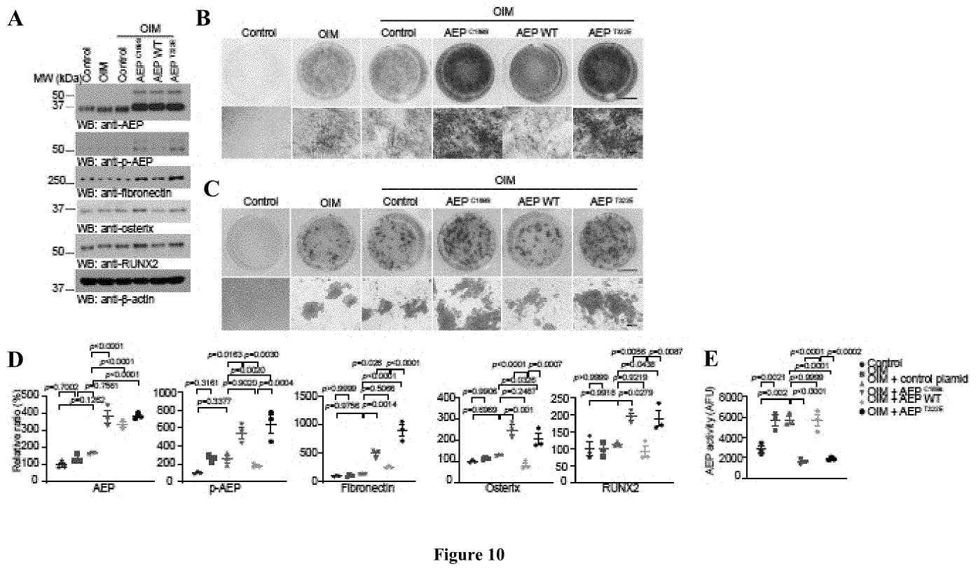

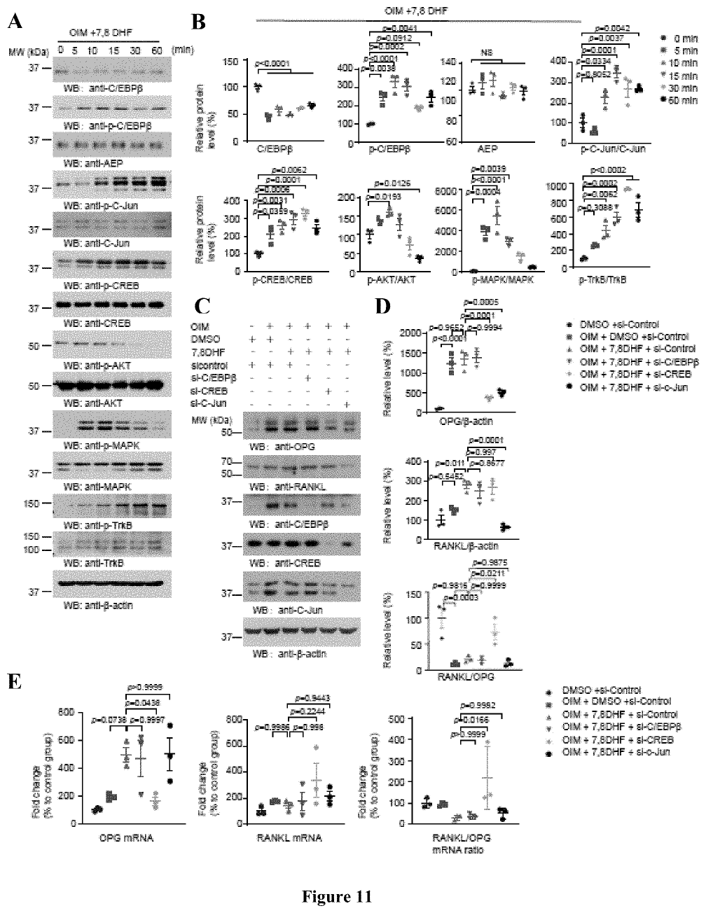

To further interrogate the role of AEP in MC3T3-E1 cell differentiation and mineralization induced by OIM, the cells with dominant-negative enzymatic-dead AEP C189S mutant, and phosphorylation mimetic AEP T322E mutant (inactive), were transferred. Blockade of AEP highly escalated fibronectin, Osterix and RUNX2, which were repressed by AEP WT (FIG. 10, Panel A and FIG. 10, Panel D). ALP staining and Alizarin Red S analysis showed that antagonizing AEP promoted osteoblast cell differentiation and bone formation (FIG. 10, Panel B and FIG. 10, Panel C). In AEP C189S and T322E mutants, but not WT, OIM-elicited AEP activities were inhibited (FIG. 10, Panel E). Quantitative RT-PCR (qRT-PCR) analysis revealed that both BDNF and 7,8-DHF exhibited strong stimulatory effect, by promoting OPG mRNA levels, which were abolished by K252a. Interestingly, the elevated RANK-L mRNAs by BDNF and 7,8-DHF were not affected by K252a (FIG. 9, Panel F). Both OPG and RANK-L protein levels were elevated by OIM in ELISA assays. These elevations were further augmented in the presence of 7,8-DHF or BDNF (FIG. 9, Panel G, left two graphs), consistent with the findings in Western blot analysis. Though both RANK-L and OPG concentrations were substantially elevated by BDNF and 7,8-DHF, the ratios of RANK-L/OPG triggered by OIM alone were significantly higher than BDNF and 7,8-DHF. Notably, K252a treatment highly augmented the ratios in BDNF and 7,8-DHF groups (FIG. 9, Panel G, left graph). Together, these observations show that 7,8-DHF mimics BDNF and that 7,8-DHF represses the C/EBPβ/AEP pathway, accelerating osteoblast formation, escalating OPG expression and decreasing the ratio of RANK-L/OPG, thereby leading to inhibition of osteoclast formation.

### Example 7: 7,8-DHF Increases OPG Expression Via Activating Transcription Factor CREB

To further interrogate the molecular mechanism of 7,8-DHF in promoting OPG expression, a time course study in MC3T3-E1 cells in the presence of OIM was conducted. Indeed, 7,8-DHF activated p-TrkB and its downstream effectors p-MAPK and p-Akt, again showing 7,8-DHF mimics BDNF by activating TrkB neurotrophic signaling. Numerous transcription factors, including c-Jun and CREB, affect OPG mRNA transcription. Noticeably, p-C/EBPβ, p-c-Jun and p-CREB signals were time-dependently increased by 7,8-DHF (FIG. 11, Panel A and FIG. 11, Panel B), suggestive of the activation of these transcription factors. To examine which of them are essential for OPG expression, each was knocked down in MC3T3-E1 cells via the specific siRNAs in the presence of OIM and 7,8-DHF. Consistently, OIM increased OPG and RANK-L, associated with C/EBPβ and c-Jun augmentation, whereas the total CREB level remained constant. Again, 7,8-DHF treatment attenuated C/EBPβ without interfering with CREB or c-Jun levels. Remarkably, knocking down CREB or c-Jun but not C/EBPβ reduced OPG protein levels, and the ratio of RANK-L/OPG was significantly augmented when CREB was depleted (FIG. 11, Panel C and FIG. 11, Panel D). qRT-PCR demonstrated that 7,8-DHF-stimulated OPG mRNA was selectively suppressed when CREB was knocked down by its siRNA, whereas RANK-L mRNA levels were similar among the groups, resulting in a higher RANK-L/OPG ratio (FIG. 11, Panel E). Hence, 7,8-DHF via activating CREB, a well-characterized downstream transcription factor of BDNF/TrkB pathway, stimulates OPG expression levels.

### Example 8: 7,8-DHF Inhibits RANK-L-Induced RAW264.7 Osteoclastogenesis

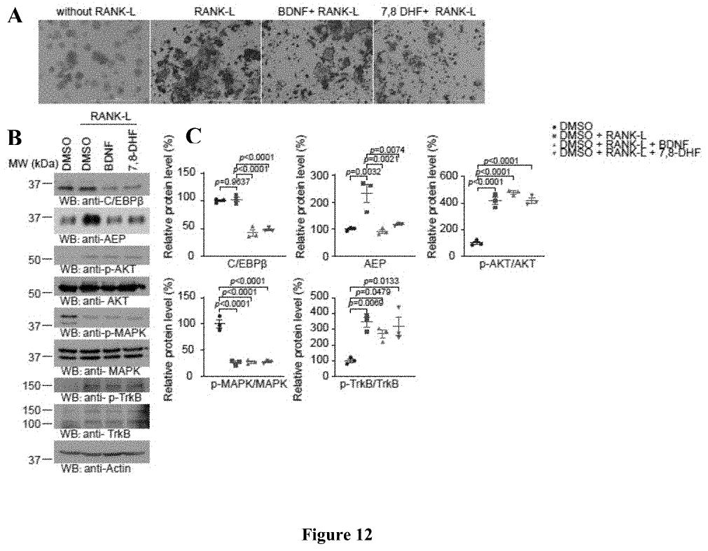

RAW264.7 cells are a well-established cellular model for osteoclastic differentiation and has been widely used in bone homeostasis research. Moreover, RANK-L independently induces RAW264.7 cell osteoclastic differentiation, which efficiently generates osteoclasts in vitro. To investigate whether the promotion of bone formation by 7,8-DHF also might involve inhibiting osteoclastogenesis, RAW264.7 cells were contacted with RANK-L. Treatment with 30 ng/ml RANK-L at day 4 significantly increased the number of multinucleated osteoclastic cells, and this increase was diminished by the addition of BDNF or 7,8-DHF, showing inhibition of RANK-L-promoted osteoclastogenesis (FIG. 12, Panel A). Immunoblotting analysis revealed that C/EBPβ was greatly reduced by 7,8-DHF or BDNF treatments, and RANK-L-stimulated AEP interrupted the upstream C/EBPβ levels (FIG. 12, Panel B and FIG. 12, Panel C). Hence, 7,8-DHF blocks RANK-L-induced RAW264.7 osteoclastogenesis associated with AEP inhibition.

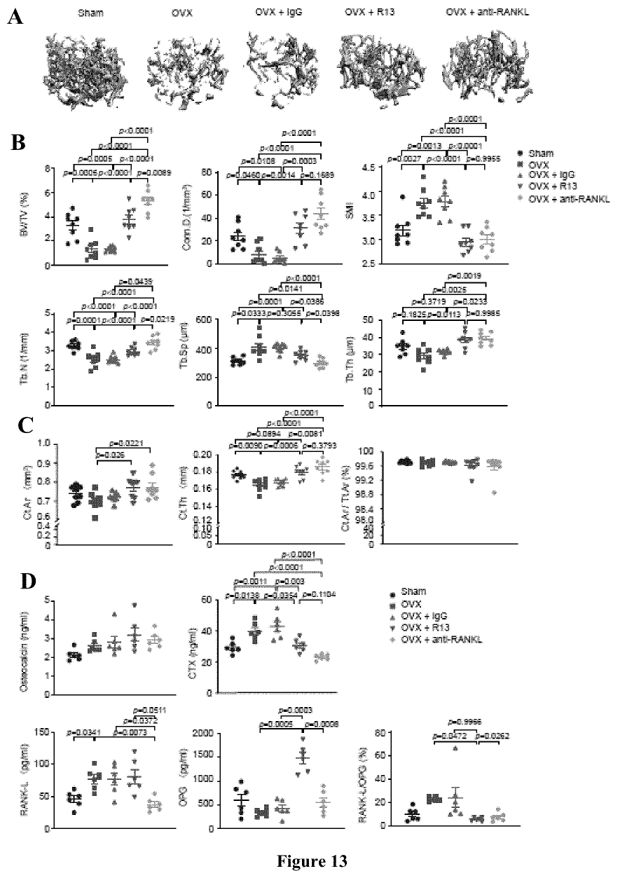

Given that R13 protects the bone by increase OPG, the RANK-L antibody was used as a comparator drug. Four weeks after ovariectomy, WT mice were treated with IgG or anti-RANK-L monoclonal antibody consecutively for 4 weeks. Remarkably, R13 displayed similar efficacy in the bone density and various bone indices to anti-RANK-L treatment (FIG. 13, Panels A-C). R13 robustly elevated OPG without altering RANK-L, whereas anti-RANK-L substantially depleted RANK-L without changing OPG, resulting in the significant reduction in the ratios of RANK-L/OPG by both treatments (FIG. 13, Panel D). Thus, R13 exhibits the same therapeutic efficacy toward osteoporosis as anti-RANK-L.

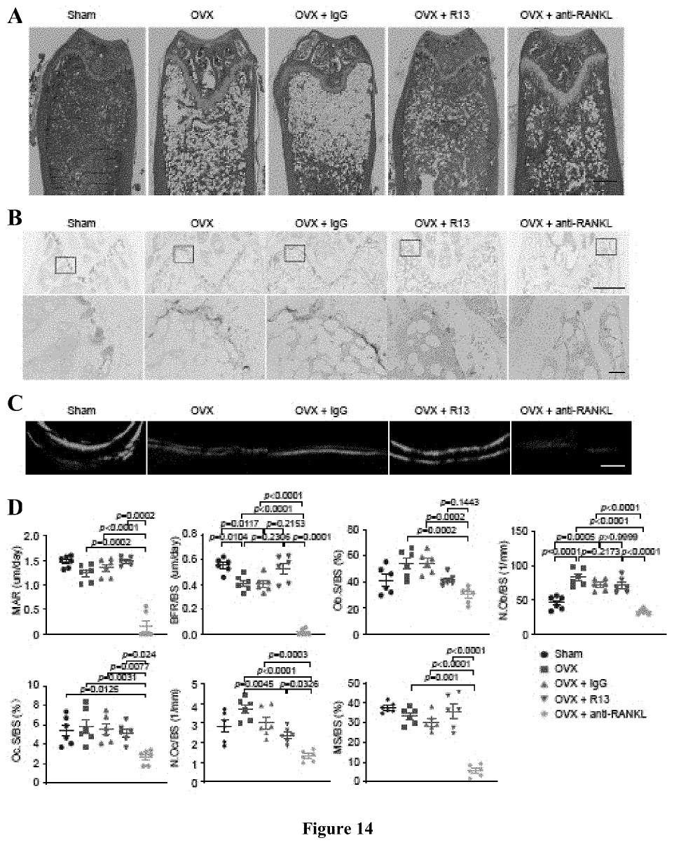

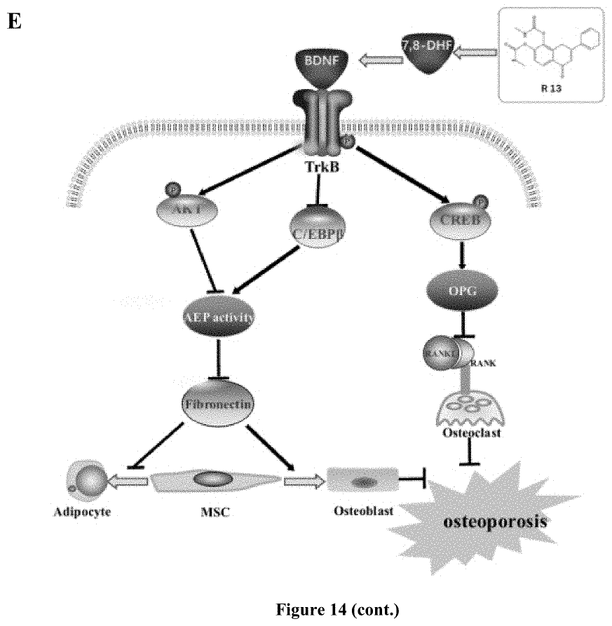

As demonstrated herein, R13 is effective for treating osteoporosis via both stimulating bone formation by enhancing osteoblast differentiation and preventing bone resorption via blocking osteoclastogenesis. R13 exerts bone protective effects via the dual mechanisms, including OPG upregulation and AEP antagonism, and can result in stronger therapeutic efficacy in patients than anti-RANK-L. H&E staining revealed that R13 treatment and anti-RANK-L increased trabecular bone marrow density and decreased the white adipocyte contents in the bone after OVX (FIG. 14, Panel A). TRAP staining demonstrated that OVX-induced osteoclast cells were diminished by both treatments (FIG. 14, Panel B). Histomorphometric analysis in the trabecular bone in the distal femur demonstrated that R13-treatment significantly increased BFR/BS and decreased N.Oc/BS in WT mice after OVX. However, anti-RANK-L antibody treatment significantly decreased the MAR, BFR/BS, ObS/BS and N.Ob/BS which are the bone formation parameters, as well as the bone resorption parameters such as OcS/BS and N.Oc/BS. Thus, R13 exhibits comparable effects to those of anti-RANK-L in reducing OVX-induced bone loss by inhibiting osteoclast (FIG. 14, Panel D). Representative data of double labeling of the trabecular bone in WT mice with OVX after R13 or anti-RANK-L antibody treatment are provided in FIG. 14, Panel C.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compositions and methods are intended to fall within the scope of the appended claims. Thus, a combination of steps, elements, components, or constituents can be explicitly mentioned herein; however, all other combinations of steps, elements, components, and constituents are included, even though not explicitly stated.

What is claimed is:

1. A method of treating or preventing a bone disorder in a subject, comprising administering to the subject an effective amount of a compound of Formula I 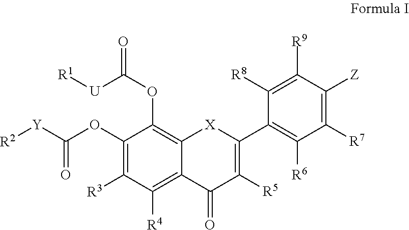
or a salt thereof, wherein:
X is O, S, or NH;
U and Y are each independently O, S, NH, Nalkyl, or CH₂;
Z is hydrogen, amino, diaminoalkyl, or heterocyclyl optionally substituted with one or more, the same or different, R¹⁵;
R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵;
R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵;
R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵;
each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and
each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

2. The method of claim 1, wherein U and Y are NH or Nalkyl.

3. The method of claim 1, wherein U and Y are O.

4. The method of claim 1, wherein R¹ and R² are alkyl.

5. The method of claim 1, wherein the compound is selected from the group consisting of
4-oxo-2-phenyl-4H-chromene-7,8-diyl bis(methylcarbamate);
4-oxo-2-phenyl-4H-chromene-7,8-diyl dipropionate;
4-oxo-2-phenyl-4H-chromene-7,8-diyl bis(2,2-dimethylpropanoate);
diethyl (4-oxo-2-phenyl-4H-chromene-7,8-diyl) dicarbonate;
4-oxo-2-phenyl-4H-chromene-7,8-diyl bis(ethylcarbamate);
4-oxo-2-phenyl-4H-chromene-7,8-diyl bis(dimethylcarbamate); and
4-oxo-2-phenyl-4H-chromene-7,8-diyl bis(3-methylbutanoate) or salts thereof.

6. The method of claim 1, wherein the bone disorder comprises a bone disorder associated with abnormally high bone catabolism.

7. The method of claim 1, wherein the bone disorder is osteoporosis, osteopenia, Paget's disease, bone metastasis, osteogenesis imperfecta, a metastatic bone disease, or a metabolic bone disease.

8. The method of claim 1, further comprising administering an additional active agent, wherein the additional active agent comprises an anti-osteoporosis agent or CF3CN.

9. The method of claim 8, wherein the anti-osteoporosis agent comprises one or more of a bisphosphonate, a RANK-L inhibitor, a parathyroid hormone, a monoclonal antibody, estrogen, and calcitonin.

10. The method of claim 1, wherein the compound is administered orally.

11. The method of claim 1, further comprising selecting a subject with or at risk of developing a bone disorder or experiencing bone loss.

12. A method of inhibiting asparagine endopeptidase (AEP) activity in a cell, comprising contacting the cell with an effective amount of a compound of Formula I 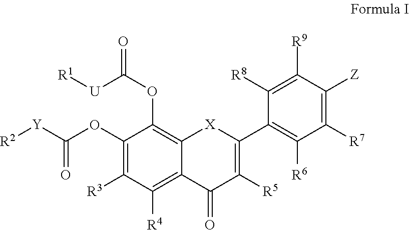
or a salt thereof, wherein:
X is O, S, or NH;
U and Y are each independently O, S, NH, Nalkyl, or CH₂;
Z is hydrogen, amino, diaminoalkyl, or heterocyclyl optionally substituted with one or more, the same or different, R¹⁵;
R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵;
R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵;
R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵;
each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and
each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

13. The method of claim 12, wherein the compound is selected from the group consisting of
4-oxo-2-phenyl-4H-chromene-7,8-diyl bis(methylcarbamate);
4-oxo-2-phenyl-4H-chromene-7,8-diyl dipropionate;
4-oxo-2-phenyl-4H-chromene-7,8-diyl bis(2,2-dimethylpropanoate);
diethyl (4-oxo-2-phenyl-4H-chromene-7,8-diyl) dicarbonate;
4-oxo-2-phenyl-4H-chromene-7,8-diyl bis(ethylcarbamate);
4-oxo-2-phenyl-4H-chromene-7,8-diyl bis(dimethylcarbamate); and
4-oxo-2-phenyl-4H-chromene-7,8-diyl bis(3-methylbutanoate) or salts thereof.

14. The method of claim 12, wherein the contacting is performed in vivo.

15. The method of claim 12, wherein the contacting is performed in vitro.

16. The method of claim 12, further comprising selecting a subject with or at risk of developing a bone disorder or experiencing bone loss.

17. The method of claim 12, wherein the contacting increases osteoprotegerin (OPG) levels in the cell as compared to an untreated cell.

18. The method of claim 12, wherein the contacting reduces trabecular bone loss induced by ovariectomy (OVX).

19. The method of claim 12, wherein the contacting inhibits RANK-L-induced osteoclastogenesis.

20. A method of promoting bone growth, increasing bone density, or increasing bone strength in a subject, comprising
administering to the subject an effective amount of a compound of Formula I 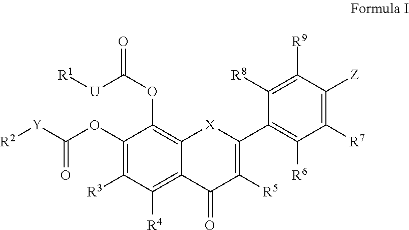
or a salt thereof, wherein:
X is O, S, or NH;
U and Y are each independently O, S, NH, Nalkyl, or CH₂;
Z is hydrogen, amino, diaminoalkyl, or heterocyclyl optionally substituted with one or more, the same or different, R¹⁵;
R¹ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, or aryl, wherein R¹ is optionally substituted with one or more, the same or different, R¹⁵;
R² is alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵;
R³, R⁴, R⁵, R⁶, R⁷, R⁸, and R⁹ are each independently hydrogen, alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R³, R⁴, R⁵, R⁶, R⁷, R⁸, and R⁹ are optionally substituted with one or more, the same or different, R¹⁵;
each R¹⁵ is independently selected from the group consisting of alkyl, halogen, nitro, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, and aryl, wherein R¹⁵ is optionally substituted with one or more, the same or different, R¹⁶; and
each R¹⁶ is independently selected from the group consisting of halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.
